(19) United States
(12) Patent Application Publication
(10) Pub. No.: US 2023/0226058 A1
(43) Pub. Date: Jul. 20, 2023

(54) METHODS AND COMPOSITIONS FOR TREATING ACUTE KIDNEY INJURY

(71) Applicant: CalciMedica, Inc., La Jolla, CA (US)

(72) Inventors: Kenneth A. STAUDERMAN, San Diego, CA (US); Michael DUNN, La Jolla, CA (US); Sudarshan HEBBAR, Kansas City, MO (US); Rachel LEHENY, La Jolla, CA (US)

(21) Appl. No.: 18/056,973

(22) Filed: Nov. 18, 2022

Related U.S. Application Data
(63) Continuation of application No. PCT/US2021/033237, filed on May 19, 2021.
(60) Provisional application No. 63/027,800, filed on May 20, 2020.

(51) Int. Cl. A61K 31/506; A61K 31/436; A61P 13/12

(52) U.S. Cl. CPC A61K 31/506; A61K 31/436; A61P 13/12

(57) ABSTRACT

Compositions and methods related to the treatment of acute kidney injury (AKI) through the pharmaceutical manipulation of calcium signaling are disclosed. Such compositions and methods may be used to reduce inflammatory responses that may lead to AKI, or the progression of AKI to CKD.

### CROSS-REFERENCE

This application is a continuation of International Application PCT/US2021/033237, filed May 19, 2021, which claims the benefit of U.S. Provisional Application No. 63/027,800, filed May 20, 2020, all of which are incorporated herein by reference in their entirety.

### BACKGROUND

Acute kidney injury (AKI), also called acute kidney failure or acute renal failure, occurs when a subject's kidneys suddenly become unable to filter waste products from the subject's blood and typically develops rapidly, usually in less than a few days. AKI affects 2%-5% of hospitalized patients and increases the risk of death in the intensive care unit (ICU), and mortality rates in this setting range between 15%-60%. Further, AKI increases the risk of adverse long-term effects, such as development of chronic kidney disease (CKD) and progression to end-stage renal disease.

Patients suffering from sepsis, blood loss, cardiac dysfunction and COVID-19, whose symptoms are severe and require hospitalization, may be at a great risk of developing AKI. Thus, there is a need to develop an effective therapeutic treatment for AKI or to prevent AKI.

### SUMMARY OF THE INVENTION

Provided herein are embodiments related to methods and compositions for reducing inflammatory responses to treat acute kidney injury (AKI).

In an aspect, the disclosure provides a method for treating AKI in a subject comprising administering a therapeutically effective amount of an intracellular Calcium signaling inhibitor to said subject.

In another aspect, the disclosure provides a method for preventing AKI in a subject at risk of developing AKI, comprising administering a prophylactically effective amount of an intracellular Calcium signaling inhibitor to said subject. In another aspect, the disclosure provides a method for preventing AKI in a subject to progress to CKD, comprising administering a prophylactically effective amount of an intracellular Calcium signaling inhibitor to said subject.

In some embodiments, the intracellular Calcium signaling inhibitor is a SOC channel inhibitor. In some embodiments, the intracellular Calcium signaling inhibitor is a CRAC channel inhibitor. In some embodiments, the intracellular Calcium signaling inhibitor inhibits a channel comprising a STIM1 protein. In some embodiments, the intracellular Calcium signaling inhibitor inhibits a channel comprising Orai1 protein. In some embodiments, the intracellular Calcium signaling inhibits a channel comprising Orai2 protein.

In some embodiments, the intracellular Calcium signaling inhibitor is a compound having a structure of:

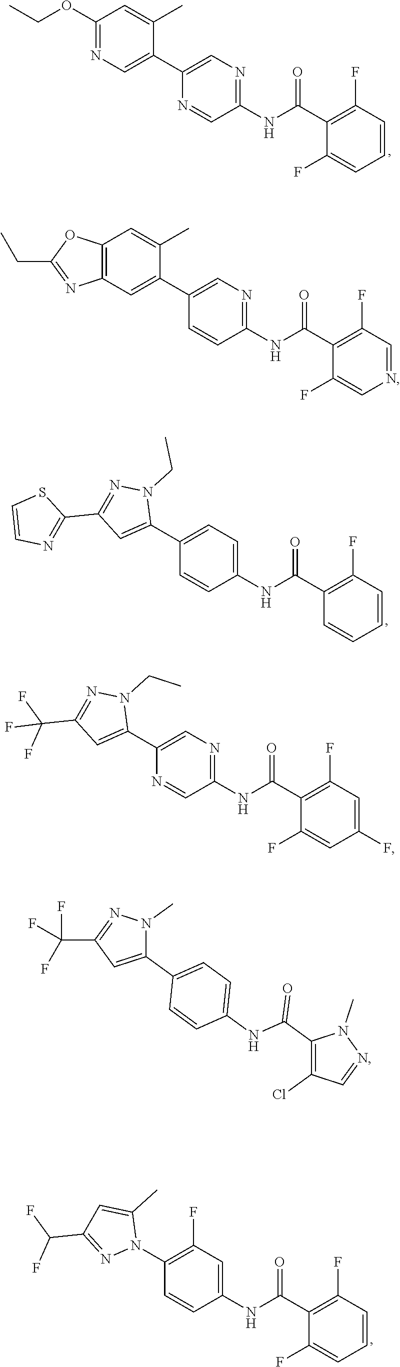 [SMILES: CCOc1cc(C)c(-c2cnc(NC(=O)c3c(F)cccc3F)cn2)cn1 ; CCc1nc2cc(-c3ccc(NC(=O)c4c(F)cncc4F)nc3)c(C)cc2o1 ; CCn1nc(-c2nccs2)cc1-c1ccc(NC(=O)c2ccccc2F)cc1 ; CCn1nc(C(F)(F)F)cc1-c1cnc(NC(=O)c2c(F)cc(F)cc2F)cn1 ; Cn1nc(C(F)(F)F)cc1-c1ccc(NC(=O)c2c(Cl)cnn2C)cc1 ; Cc1cc(C(F)F)nn1-c1ccc(NC(=O)c2c(F)cccc2F)cc1F]

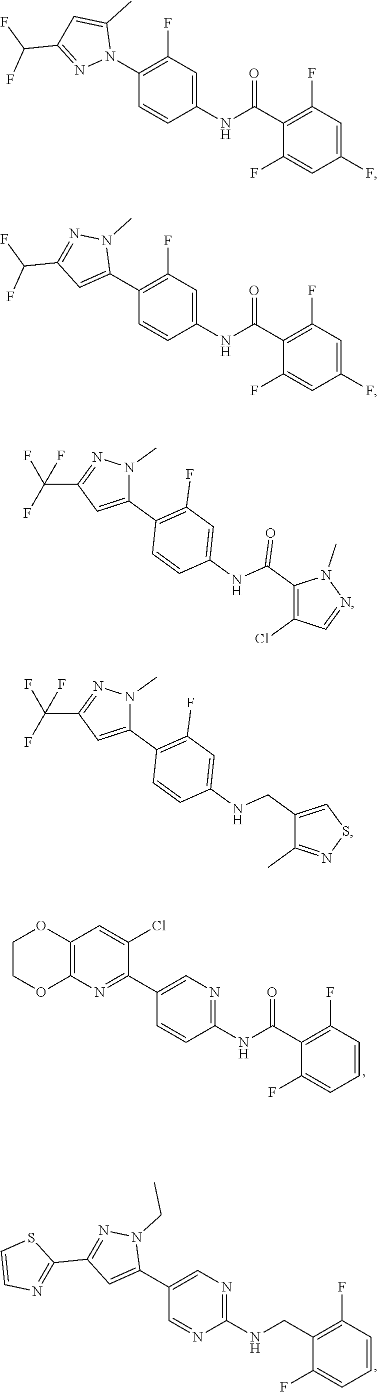 [SMILES: Cc1cc(C(F)F)nn1-c1ccc(NC(=O)c2c(F)cc(F)cc2F)cc1F ; Cn1nc(C(F)F)cc1-c1ccc(NC(=O)c2c(F)cc(F)cc2F)cc1F ; Cn1nc(C(F)(F)F)cc1-c1ccc(NC(=O)c2c(Cl)cnn2C)cc1F ; Cc1nscc1CNc1ccc(-c2cc(C(F)(F)F)nn2C)c(F)c1 ; O=C(Nc1ccc(-c2nc3c(cc2Cl)OCCO3)cn1)c1c(F)cccc1F ; CCn1nc(-c2nccs2)cc1-c1cnc(NCc2c(F)cccc2F)nc1]

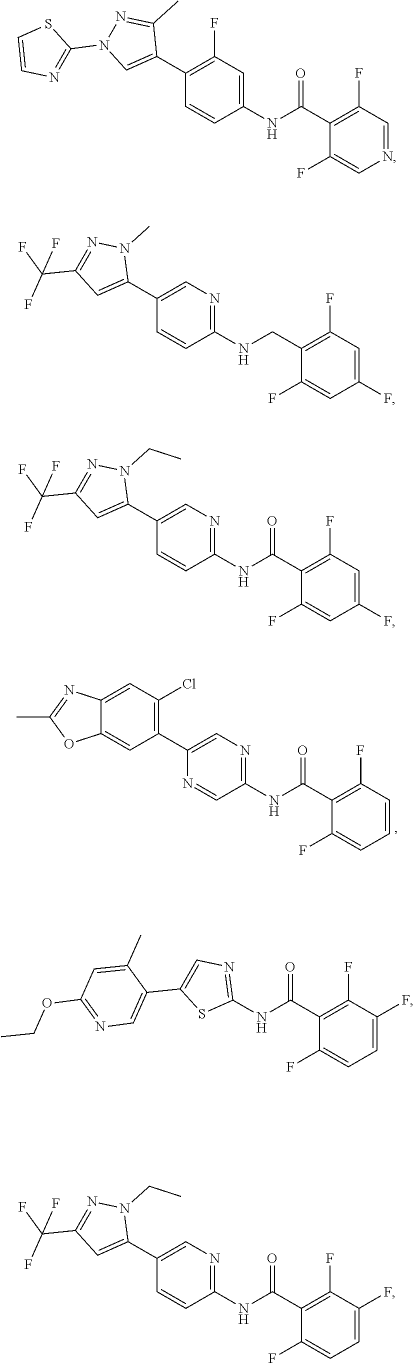 [SMILES: Cc1nn(-c2nccs2)cc1-c1ccc(NC(=O)c2c(F)cncc2F)cc1F ; Cn1nc(C(F)(F)F)cc1-c1ccc(NCc2c(F)cc(F)cc2F)nc1 ; CCn1nc(C(F)(F)F)cc1-c1ccc(NC(=O)c2c(F)cc(F)cc2F)nc1 ; Cc1nc2cc(Cl)c(-c3cnc(NC(=O)c4c(F)cccc4F)cn3)cc2o1 ; CCOc1cc(C)c(-c2cnc(NC(=O)c3c(F)ccc(F)c3F)s2)cn1 ; CCn1nc(C(F)(F)F)cc1-c1ccc(NC(=O)c2c(F)ccc(F)c2F)nc1]

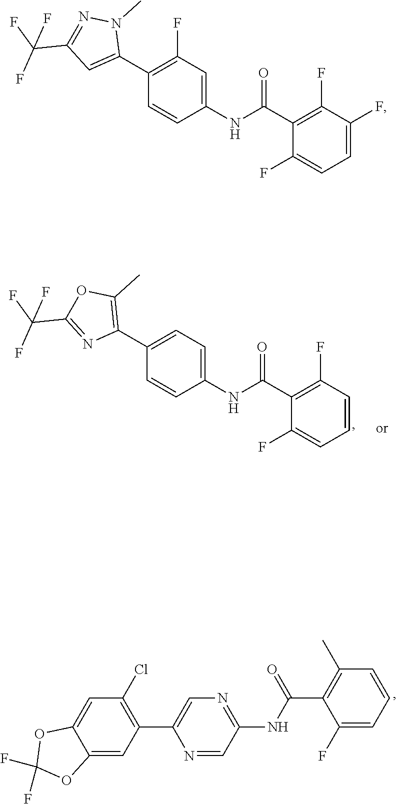 [SMILES: Cn1nc(C(F)(F)F)cc1-c1ccc(NC(=O)c2c(F)ccc(F)c2F)cc1F ; Cc1oc(C(F)(F)F)nc1-c1ccc(NC(=O)c2c(F)cccc2F)cc1 ; Cc1cccc(F)c1C(=O)Nc1cnc(-c2cc3c(cc2Cl)OC(F)(F)O3)cn1]

(collectively, “Compound A”, or a pharmaceutically acceptable salt, pharmaceutically acceptable solvate, or pharmaceutically acceptable prodrug thereof. In some embodiments the intracellular Calcium signaling inhibitor is a compound having a structure from the group of Compound A or a nanoparticle formulation thereof, including a nanoparticle suspension or emulsion.

In some embodiments, the intracellular Calcium signaling inhibitor is a compound of N-(5-(6-chloro-2,2-difluorobenzo[d][1,3]dioxol-5-yl)pyrazin-2-yl)-2-fluoro-6-methylbenzamide. In some aspects the intracellular Calcium signaling inhibitor is a compound of N-(5-(6-chloro-2,2-difluorobenzo[d][1,3]dioxol-5-yl)pyrazin-2-yl)-2-fluoro-6-methylbenzamide or a pharmaceutically acceptable salt, pharmaceutically acceptable solvate, or pharmaceutically acceptable prodrug thereof. In some aspects the intracellular Calcium signaling inhibitor is chosen from among the compounds, N-(5-(6-ethoxy-4-methylpyridin-3-yl)pyrazin-2-yl)-2,6-difluorobenzamide, N-(5-(2-ethyl-6-methylbenzo[d]oxazol-5-yl)pyridin-2-yl)-3,5-difluoroisonicotinamide, N-(4-(1-ethyl-3-(thiazol-2-yl)-1H-pyrazol-5-yl)phenyl)-2-fluorobenzamide, N-(5-(1-ethyl-3-(triflouromethyl)-1H-pyrazol-5-yl)pyrazin-2-yl)-2,4,6-trifluorobenzamide, 4-chloro-1-methyl-N-(4-(1-methyl-3-(trifluoromethyl)-1H-pyrazol-5-yl)phenyl)-1H-pyrazole-5-carboxamide, N-(4-(3-(difluoromethyl)-5-methyl-1H-pyrazol-1-yl)-3-fluorophenyl)-2,6-difluorobenzamide, N-(4-(3-(difluoromethyl)-5-methyl-1H-pyrazol-1-yl)-3-fluorophenyl)-2,4,6-trifluorobenzamide, N-(4-(3-(difluoromethyl)-1-methyl-1H-pyrazol-5-yl)-3-fluorophenyl)-2,4,6-trifluorobenzamide, 4-chloro-N-(3-fluoro-4-(1-methyl-3-(trifluoromethyl)-1H-pyrazol-5-yl)phenyl)-1-methyl-1H-pyrazole-5-carboxamide, 3-fluoro-4-(1-methyl-3-(trifluoromethyl)-1H-pyrazol-5-yl)-N-((3-methylisothiazol-4-yl)methyl)aniline, N-(5-(7-chloro-2,3-dihydro-[1,4]dioxino[2,3-b]pyridin-6-yl)pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl)isonicotinamide, 5-(1-methyl-3-(trifluoromethyl)-1H-pyrazol-5-yl)-N-(2,4,6-trifluorobenzyl)pyridin-2-amine, N-(5-(1-ethyl-3-(trifluoromethyl)-1H-pyrazol-5-yl)pyridin-2-yl)-2,4,6-trifluorobenzamide, N-(5-(5-chloro-2-methylbenzo[d]oxazol-6-yl)pyrazin-2-yl)-2,6-difluorobenzamide, N-(5-(6-ethoxy-4-methylpyridin-3-yl)thiazol-2-yl)-2,3,6-trifluorobenzamide, N-(5-(1-ethyl-3-(trifluoromethyl)-1H-pyrazol-5-yl)pyridin-2-yl)-2,3,6-trifluorobenzamide, 2,3,6-trifluoro-N-(3-fluoro-4-(1-methyl-3-(trifluoromethyl)-1H-pyrazol-5-yl)phenyl)benzamide, 2,6-difluoro-N-(4-(5-methyl-2-(trifluoromethyl)oxazol-4-yl)phenyl)benzamide, or N-(5-(6-chloro-2,2-difluorobenzo[d][1,3]dioxol-5-yl)pyrazin-2-yl)-2-fluoro-6-methylbenzamide, (collectively, “Compound A”), or a pharmaceutically acceptable salt, pharmaceutically acceptable solvate, or pharmaceutically acceptable prodrug thereof.

In some embodiments, the intracellular Calcium signaling inhibitor is a compound of chemical name N-(5-(6-Chloro-2,2-difluorobenzo[d][1,3]dioxol-5-yl)pyrazin-2-yl)-2-fluoro-6-methylbenzamide or a pharmaceutically acceptable salt, pharmaceutically acceptable solvate, or pharmaceutically acceptable prodrug thereof.

In some embodiments, the intracellular Calcium signaling inhibitor is a compound of chemical name 2,6-Difluoro-N-(1-(4-hydroxy-2-(trifluoromethyl)benzyl)-1H-pyrazol-3-yl)benzamide or a pharmaceutically acceptable salt, pharmaceutically acceptable solvate, or pharmaceutically acceptable prodrug thereof.

In another aspect, the disclosure herein provides a composition comprising an intracellular Calcium signaling inhibitor and at least a compound for treating acute kidney injury (AKI). In some embodiments, the compound is selected from the list consisting of a recombinant human IGF-I (rhIGF-I), atrial natriuretic peptide (ANP), dopamine, caspase inhibitor, minocycline, guanosine and Pifithrin-α(p53 Inhibitor), poly ADP-ribose polymerase inhibitor, deferoxamine, ethyl pyruvate, activated protein C, insulin, recombinant erythropoietin, hepatocyte growth factor, carbon monoxide release compound, bilirubin, endothelin antagonist, sphingosine 1 phosphate analog, adenosine analog, inducible nitric oxide synthase inhibitor, fibrate, neutrophil gelatinase-associated lipocalin, IL-6 antagonist, C5a antagonist, IL-10, dexmedetomidine, a chloroquine (CQ), hydroxychloroquine (HCQ), nd α-melanocyte-stimulating hormone.

In another aspect, the disclosure herein provides a dosing regimen comprising administration to a subject of a compound for treating AKI, and administration of an intracellular Calcium signaling inhibitor.

In another aspect, the disclosure herein provides a composition for preventing AKI in a subject at risk of developing AKI, comprising administering a prophylactically effective amount of an intracellular Calcium signaling inhibitor.

In another aspect, the disclosure herein provides a composition for preventing AKI to progress to chronic kidney disease (CKD) in a subject who already has developed AKI, comprising administering a prophylactically effective amount of an intracellular Calcium signaling inhibitor.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates that in predicted severe acute pancreatitis (AP) patients (SIRS+ with SpO2<96%), the compound/composition disclosed herein reduced percentage of patients with de novo acute kidney injury during their hospitalization over historic and study SOC controls. Percent of patients developing AKI is 8% when the patients received treatment of the compound/composition disclosed herein. Percent of the two groups of patients developing AKI that did not receive treatment of the compound/composition disclosed herein are 50% and 20%, respectively.

### DETAILED DESCRIPTION OF THE INVENTION

Methods and compositions disclosed herein are used for modulating intracellular calcium to treat or prevent acute kidney injury (AKI), including its progression to chronic kidney injury (CKD). In some aspects, compounds provided herein modulate SOC channel activity. In some aspects, methods and compounds provided herein modulate CRAC channel activity. In another aspect, compounds provided herein modulate STIM protein activity. In another aspect, methods and compounds provided herein modulate Orai protein activity. In another aspect, methods and compounds provided herein modulate the functional interactions of STIM proteins with Orai proteins. In another aspect, methods and compounds provided herein reduce the number of functional SOC channels. In another aspect, methods and compounds provided herein reduce the number of functional CRAC channels. In some aspects, methods and compounds described herein are SOC channel blockers. In some aspects, methods and compounds described herein are CRAC channel blockers or CRAC channel modulators.

Calcium plays a vital role in cell function and survival. Specifically, calcium is a key element in the transduction of signals into and within cells. Cellular responses to growth factors, neurotransmitters, hormones and a variety of other signal molecules are initiated through calcium-dependent processes.

Almost all cell types depend in some manner upon the generation of cytoplasmic Ca²⁺ signals to regulate cell function, or to trigger specific responses. Cytosolic Ca²⁺ signals control a wide array of cellular functions ranging from short-term responses such as contraction and secretion to longer-term regulation of cell growth and proliferation. Usually, these signals involve some combination of release of Ca²⁺ from intracellular stores, such as the endoplasmic reticulum (ER), and influx of Ca²⁺ across the plasma membrane. In one example, cell activation begins with an agonist binding to a surface membrane receptor, which is coupled to phospholipase C (PLC) through a G-protein mechanism. PLC activation leads to the production of inositol 1,4,5-triphosphate (P), which in turn activates the IP₃ receptor causing release of Ca²⁺ from the ER. The fall in ER Ca²⁺ then signals to activate plasma membrane store-operated calcium (SOC) channels.

Store-operated calcium (SOC) influx is a process in cellular physiology that controls such diverse functions such as, but not limited to, refilling of intracellular Ca²⁺ stores (Putney et al. Cell, 75, 199-201, 1993), activation of enzymatic activity (Fagan et al., J. Biol. Chem. 275:26530-26537, 2000), gene transcription (Lewis, Annu. Rev. Immunol. 19:497-521, 2001), cell proliferation (Nunez et al., J. Physiol. 571.1, 57-73, 2006), and release of cytokines (Winslow et al., Curr. Opin. Immunol. 15:299-307, 2003). In some nonexcitable cells, e.g., blood cells, immune cells, hematopoietic cells, T lymphocytes and mast cells, pancreatic acinar cells (PACs), epithelial and ductal cells of other glands (e.g. salivary glands), endothelial and endothelial progenitor cells, SOC influx occurs through calcium release-activated calcium (CRAC) channels, a type of SOC channel.

The calcium influx mechanism has been referred to as store-operated calcium entry (SOCE). Stromal interaction molecule (STIM) proteins are an essential component of SOC channel function, serving as the sensors for detecting the depletion of calcium from intracellular stores and for activating SOC channels.

### Calcium Homeostasis

Cellular calcium homeostasis is a result of the summation of regulatory systems involved in the control of intracellular calcium levels and movements. Cellular calcium homeostasis is achieved, at least in part, by calcium binding and by movement of calcium into and out of the cell across the plasma membrane and within the cell by movement of calcium across membranes of intracellular organelles including, for example, the endoplasmic reticulum, sarcoplasmic reticulum, mitochondria and endocytic organelles including endosomes and lysosomes.

Movement of calcium across cellular membranes is carried out by specialized proteins. For example, calcium from the extracellular space can enter the cell through various calcium channels and a sodium/calcium exchanger and is actively extruded from the cell by calcium pumps and sodium/calcium exchangers. Calcium can also be released from internal stores through inositol trisphosphate or ryanodine receptors and can be taken up by these organelles by means of calcium pumps.

Calcium can enter cells by any of several general classes of channels, including but not limited to, voltage-operated calcium (VOC) channels, ligand-gated calcium channels, store-operated calcium (SOC) channels, and sodium/calcium exchangers operating in reverse mode. VOC channels are activated by membrane depolarization and are found in excitable cells like nerve and muscle and are for the most part not found in nonexcitable cells. Under some conditions, Ca²⁺ can enter cells via Na⁺—Ca²⁺ exchangers operating in reverse mode.

Endocytosis provides another process by which cells can take up calcium from the extracellular medium through endosomes. In addition, some cells, e.g., exocrine cells, can release calcium via exocytosis.

Cytosolic calcium concentration is tightly regulated with resting levels usually estimated at approximately 0.1 μM in mammalian cells, whereas the extracellular calcium concentration is typically about 2 mM. This tight regulation facilitates transduction of signals into and within cells through transient calcium flux across the plasma membrane and membranes of intracellular organelles. There is a multiplicity of intracellular calcium transport and buffer systems in cells that serve to shape intracellular calcium signals and maintain the low resting cytoplasmic calcium concentration. In cells at rest, the principal components involved in maintaining basal calcium levels are calcium pumps and leak pathways in both the endoplasmic reticulum and plasma membrane. Disturbance of resting cytosolic calcium levels can affect transmission of calcium-dependent signals and give rise to defects in a number of cellular processes. For example, cell proliferation involves a prolonged calcium signaling sequence. Other cellular processes that involve calcium signaling include, but are not limited to, secretion, transcription factor signaling, and fertilization.

Cell-surface receptors that activate phospholipase C (PLC) create cytosolic Ca²⁺ signals from intra- and extra-cellular sources. An initial transient rise of [Ca²⁺]i (intracellular calcium concentration) results from the release of Ca²⁺ from the endoplasmic reticulum (ER), which is triggered by the PLC product, inositol-1,4,5-trisphosphate (IP₃), opening IP₃ receptors in the ER (Streb et al. Nature, 306, 67-69, 1983). A subsequent phase of sustained Ca²⁺ entry across the plasma membrane then ensues, through specialized store operated calcium (SOC) channels (in the case of non-excitable cells like immune PAC cells, the SOC channels are calcium release-activated calcium (CRAC) channels) in the plasma membrane. Store-operated Ca2+ entry (SOCE) is the process in which the emptying of Ca²⁺ stores itself activates Ca²⁺ channels in the plasma membrane to help refill the stores (Putney, Cell Calcium, 7, 1-12, 1986; Parekh et al., Physiol. Rev. 757-810; 2005). SOCE does more than simply provide Ca2+ for refilling stores, but can itself generate sustained Ca²⁺ signals that control such essential functions as gene expression, cell metabolism and exocytosis (Parekh and Putney, Physiol. Rev. 85, 757-810 (2005).

In lymphocytes and mast cells, activation of antigen or Fc receptors, respectively causes the release of Ca²⁺ from intracellular stores, which in turn leads to Ca²⁺ influx through CRAC channels in the plasma membrane. The subsequent rise in intracellular Ca²⁺ activates calcineurin, a phosphatase that regulates the transcription factor NFAT. In resting cells, NFAT is phosphorylated and resides in the cytoplasm, but when dephosphorylated by calcineurin, NFAT translocates to the nucleus and activates different genetic programs depending on stimulation conditions and cell type. In response to infections and during transplant rejection, NFAT partners with the transcription factor AP-1 (Fos-Jun) in the nucleus of “effector” T cells, thereby trans-activating cytokine genes, genes that regulate T cell proliferation and other genes that orchestrate an active immune response (Rao et al., Annu Rev Immunol., 1997; 15:707-47). In contrast, in T cells recognizing self-antigens, NFAT is activated in the absence of AP-1, and activates a transcriptional program known as “anergy” that suppresses autoimmune responses (Macian et al., Transcriptional mechanisms underlying lymphocyte tolerance. Cell. 2002 Jun. 14; 109(6):719-31). In a subclass of T cells known as regulatory T cells which suppress autoimmunity mediated by self-reactive effector T cells, NFAT partners with the transcription factor FOXP3 to activate genes responsible for suppressor function (Wu et al., Cell, 2006 Jul. 28; 126(2):375-87; Rudensky A Y, Gavin M, Zheng Y. Cell. 2006 Jul. 28; 126(2):253-256). Another subclass of T cells is T-helper 17 (Th17) cells, a unique CD4+ T-cell subset characterized by production of interleukin-17 (IL-17). Recent data in humans and mice suggest that Th17 cells play an important role in the pathogenesis of a diverse group of immune-mediated diseases, including, acute kidney injury, psoriasis, rheumatoid arthritis, multiple sclerosis, inflammatory bowel disease, and asthma. Th17 cells also play an important role in a subject progressing from AKI to chronic kidney disease (CKD).

The endoplasmic reticulum (ER) carries out a variety of processes. The ER has a role as both a Ca²⁺ sink and an agonist-sensitive Ca²⁺ store, and protein folding/processing takes place within its lumen. In the latter case, numerous Ca²⁺-dependent chaperone proteins ensure that newly synthesized proteins are folded correctly and sent off to their appropriate destination. The ER is also involved in vesicle trafficking, release of stress signals, regulation of cholesterol metabolism, and apoptosis. Many of these processes require intraluminal Ca²⁺ and protein misfolding, ER stress responses, and apoptosis can all be induced by depleting the ER of Ca²⁺ for prolonged periods of time. Because it contains a finite amount of Ca²⁺, it is clear that ER Ca²⁺ content must fall after release of that Ca²⁺ during stimulation. However, to preserve the functional integrity of the ER, it is vital that the Ca²⁺ content does not fall too low or is maintained at least at a low level. Replenishment of the ER with Ca²⁺ is therefore a central process to all eukaryotic cells. Because a fall in ER Ca²⁺ content activates store-operated Ca²⁺ channels in the plasma membrane, a major function of this Ca²⁺ entry pathway is believed to be maintenance of ER Ca²⁺ levels that are necessary for proper protein synthesis and folding. However, store-operated Ca²⁺ channels have other important roles.

The understanding of store-operated calcium entry was provided by electrophysiological studies which established that the process of emptying the stores activated a Ca²⁺ current in mast cells called Ca²⁺ release-activated Ca²⁺ current or ICRAC. ICRAC is non-voltage activated, inwardly rectifying, and remarkably selective for Ca²⁺. It is found in several cell types mainly of hemapoietic origin. ICRAC is not the only store-operated current, and it is now apparent that store-operated influx encompasses a family of Ca²⁺-permeable channels, with different properties in different cell types. ICRAC was the first store-operated Ca²⁺ current to be described and remains a popular model for studying store-operated influx.

Store-operated calcium channels can be activated by any procedure that empties ER Ca²⁺ stores; it does not seem to matter how the stores are emptied, the net effect is activation of store-operated Ca²⁺ entry. Physiologically, store emptying is evoked by an increase in the levels of IP₃ or other Ca²⁺-releasing signals followed by Ca²⁺ release from the stores. But there are several other methods for emptying stores. These methods include the following:
1) elevation of IP₃ in the cytosol (following receptor stimulation or, dialyzing the cytosol with IP₃ itself or related congeners like the nonmetabolizable analog Ins (2,4,5)P₃);
2) application of a Ca²⁺ ionophore (e.g., ionomycin) to permeabilize the ER membrane;
3) dialyzing the cytoplasm with high concentrations of Ca²⁺ chelators (e.g., EGTA or BAPTA), which chelate Ca²⁺ that leaks from the stores and hence prevent store refilling;
4) exposure to the sarcoplasmic/endoplasmic reticulum Ca²⁺-ATPase (SERCA) inhibitors like thapsigargin, cyclopiazonic acid, and di-tert-butylhydroquinone;
5) sensitizing the IP₃ receptors to resting levels of InsP3 with agents like thimerosal; and
6) loading membrane-permeable metal Ca²⁺ chelators like N,N,N′,N′-tetrakis(2-pyridylmethyl)ethylene diamine (TPEN) directly into the stores.
   Through mass action, TPEN lowers free intraluminal Ca²⁺ concentration without changing total store Ca²⁺ such that the store depletion-dependent signal is generated.

These methods of emptying stores are not devoid of potential problems. The key feature of store-operated Ca²⁺ entry is that it is the fall in Ca²⁺ content within the stores and not the subsequent rise in cytoplasmic Ca²⁺ concentration that activates the channels. However, ionomycin and SERCA pump blockers generally cause a rise in cytoplasmic Ca²⁺ concentration as a consequence of store depletion, and such a rise in Ca²⁺ could open Ca²⁺-activated cation channels permeable to Ca²⁺. One way to avoid such problems is to use agents under conditions where cytoplasmic Ca²⁺ has been strongly buffered with high concentrations of Ca²⁺ chelator such as EGTA or BAPTA.

### Store-Operated Calcium Entry

Reduced calcium concentration in intracellular calcium stores such as the endoplasmic reticulum resulting from release of calcium therefrom provides a signal for influx of calcium from the extracellular medium into the cell. This influx of calcium, which produces a sustained “plateau” elevation of cytosolic calcium concentration, generally does not rely on voltage-gated plasma membrane channels and does not involve activation of calcium channels by calcium. This calcium influx mechanism is referred to as capacitive calcium entry (CCE), calcium release-activated, store-operated or depletion-operated calcium entry. Store-operated calcium entry can be recorded as an ionic current with distinctive properties. This current is referred to as I_{SOC} (store-operated current) or I_{CRAC} (calcium release-activated current).

Electrophysiological analysis of store-operated or calcium release-activated currents reveal distinct biophysical properties (see, e.g., Parekh and Penner (1997) Physiol. Rev. 77:901-930) of these currents. For example, the current can be activated by depletion of intracellular calcium stores (e.g., by non-physiological activators such as thapsigargin, CPA, ionomycin and BAPTA, and physiological activators such as IP₃) and can be selective for divalent cations, such as calcium, over monovalent ions in physiological solutions or conditions, can be influenced by changes in cytosolic calcium levels, and can show altered selectivity and conductivity in the presence of low extracellular concentrations of divalent cations. The current may also be blocked or enhanced by 2-APB (depending on concentration) and blocked by SKF96365 and Gd³⁺ and generally can be described as a calcium current that is not strictly voltage-gated.

Patch-clamp studies in mast cells and Jurkat leukemic T cells have established the CRAC entry mechanism as an ion channel with distinctive biophysical characteristics, including a high selectivity for Ca²⁺ paired with an exceedingly low conductance. Furthermore, the CRAC channel was shown to fulfill the rigorous criteria for being store-operated, which is the activation solely by the reduction of Ca²⁺ in the ER rather than by cytosolic Ca²⁺ or other messengers generated by PLC (Prakriya et al., In Molecular and Cellular Insights into Ion Channel Biology (ed. Robert Maue) 121-140 (Elsevier Science, Amsterdam, 2004)).

### Regulation of Store-Operated Calcium Entry by Intracellular Calcium Stores

Store-operated calcium entry is regulated by the level of calcium within an intracellular calcium store. Intracellular calcium stores can be characterized by sensitivity to agents, which can be physiological or pharmacological, which activate release of calcium from the stores or inhibit uptake of calcium into the stores. Different cells have been studied in characterization of intracellular calcium stores, and stores have been characterized as sensitive to various agents, including, but not limited to, IP₃ and compounds that effect the IP₃ receptor, thapsigargin, ionomycin and/or cyclic ADP-ribose (cADPR)(see, e.g., Berridge (1993) Nature 361:315-325; Churchill and Louis (1999) Am. J. Physiol. 276: C₄26-C₄34; Dargie et al. (1990) Cell Regul. 1:279-290; Gerasimenko et al. (1996) Cell 84:473-480; Gromoda et al. (1995) FEBS Lett. 360:303-306; Guse et al. (1999) Nature 398:70-73).

Accumulation of calcium within endoplasmic reticulum and sarcoplasmic reticulum (SR; a specialized version of the endoplasmic reticulum in striated muscle) storage organelles is achieved through sarcoplasmic-endoplasmic reticulum calcium ATPases (SERCAs), commonly referred to as calcium pumps. During signaling (i.e., when endoplasmic reticulum channels are activated to provide for calcium release from the endoplasmic reticulum into the cytoplasm), endoplasmic reticulum calcium is replenished by the SERCA pump with cytoplasmic calcium that has entered the cell from the extracellular medium (Yu and Hinkle (2000) J. Biol. Chem. 275:23648-23653; Hofer et al. (1998) EMBO J. 17:1986-1995).

Calcium release channels associated with IP₃ and ryanodine receptors provide for controlled release of calcium from endoplasmic and sarcoplasmic reticulum into the cytoplasm resulting in transient increases in cytoplasmic calcium concentration. IP₃ receptor-mediated calcium release is triggered by IP₃ formed by the breakdown of plasma membrane phosphoinositides through the action of phospholipase C, which is activated by binding of an agonist to a plasma membrane G protein-coupled receptor or tyrosine kinase. Ryanodine receptor-mediated calcium release is triggered by an increase in cytoplasmic calcium and is referred to as calcium-induced calcium release (CICR). The activity of ryanodine receptors (which have affinity for ryanodine and caffeine) may also be regulated by cyclic ADP-ribose.

Thus, the calcium levels in the stores, and in the cytoplasm, fluctuate. For example, ER free calcium concentration can decrease from a range of about 60-400 μM to about 1-50 μM when HeLa cells are treated with histamine, an agonist of PLC-linked histamine receptors (Miyawaki et al. (1997) Nature 388:882-887). Store-operated calcium entry is activated as the free calcium concentration of the intracellular stores is reduced. Depletion of store calcium, as well as a concomitant increase in cytosolic calcium concentration, can thus regulate store-operated calcium entry into cells.

### Cytoplasmic Calcium Buffering

Agonist activation of signaling processes in cells can involve dramatic increases in the calcium permeability of the endoplasmic reticulum, for example, through opening of IP₃ receptor channels, and the plasma membrane through store-operated calcium entry. These increases in calcium permeability are associated with an increase in cytosolic calcium concentration that can be separated into two components: a “spike” of calcium release from the endoplasmic reticulum during activation of the IP₃ receptor and a plateau phase which is a sustained elevation of calcium levels resulting from entry of calcium into the cytoplasm from the extracellular medium. Upon stimulation, the resting intracellular free calcium concentration of about 100 nM can rise globally to greater than 1 μM and higher in microdomains of the cell. The cell modulates these calcium signals with endogenous calcium buffers, including physiological buffering by organelles such as mitochondria, endoplasmic reticulum and Golgi. Mitochondrial uptake of calcium through a uniporter in the inner membrane is driven by the large negative mitochondrial membrane potential, and the accumulated calcium is released slowly through sodium-dependent and -independent exchangers, and, under some circumstances, the permeability transition pore (PTP). Thus, mitochondria can act as calcium buffers by taking up calcium during periods of cellular activation and can slowly release it later. Uptake of calcium into the endoplasmic reticulum is regulated by the sarcoplasmic and endoplasmic reticulum calcium ATPase (SERCA). Uptake of calcium into the Golgi is mediated by a P-type calcium transport ATPase (PMR₁/ATP2C₁). Additionally, there is evidence that a significant amount of the calcium released upon IP₃ receptor activation is extruded from the cell through the action of the plasma membrane calcium ATPase. For example, plasma membrane calcium ATPases provide the dominant mechanism for calcium clearance in human T cells and Jurkat cells, although sodium/calcium exchange also contributes to calcium clearance in human T cells. Within calcium-storing organelles, calcium ions can be bound to specialized calcium-buffering proteins, such as, for example, calsequestrins, calreticulins and calnexins. Additionally, there are calcium-buffering proteins in the cytosol that modulate calcium spikes and assist in redistribution of calcium ions. Thus, proteins and other molecules that participate in any of these and other mechanisms through which cytosolic calcium levels can be reduced are proteins that are involved in, participate in and/or provide for cytoplasmic calcium buffering. Thus, cytoplasmic calcium buffering helps regulate cytoplasmic Ca²⁺ levels during periods of sustained calcium influx through SOC channels or bursts of Ca²⁺ release. Large increases in cytoplasmic Ca²⁺ levels or store refilling deactivate SOCE.

### Downstream Calcium Entry-Mediated Events

In addition to intracellular changes in calcium stores, store-operated calcium entry affects a multitude of events that are consequent to or in addition to the store-operated changes. For example Ca²⁺ influx results in the activation of a large number of calmodulin-dependent enzymes including the serine phosphatase calcineurin. Activation of calcineurin by an increase in intracellular calcium results in acute secretory processes such as mast cell degranulation. Activated mast cells release preformed granules containing histamine, heparin, TNFα and enzymes such as s-hexosaminidase. Some cellular events, such as B and T cell proliferation, require sustained calcineurin signaling, which requires a sustained increase in intracellular calcium. A number of transcription factors are regulated by calcineurin, including NFAT (nuclear factor of activated T cells), MEF₂ and NFκB. NFAT transcription factors play important roles in many cell types, including immune cells. In immune cells NFAT mediates transcription of a large number of molecules, including cytokines, chemokines and cell surface receptors. Transcriptional elements for NFAT have been found within the promoters of cytokines such as IL-2, IL-3, IL-4, IL-5, IL-8, IL-13, IL-17 as well as tumor necrosis factor alpha (TNFα), granulocyte colony-stimulating factor (G-CSF), and gamma-interferon (γ-IFN).

The activity of NFAT proteins is regulated by their phosphorylation level, which in turn is regulated by both calcineurin and NFAT kinases. Activation of calcineurin by an increase in intracellular calcium levels results in dephosphorylation of NFAT and entry into the nucleus. Rephosphorylation of NFAT masks the nuclear localization sequence of NFAT and prevents its entry into the nucleus. Because of its strong dependence on calcineurin-mediated dephosphorylation for localization and activity, NFAT is a sensitive indicator of intracellular free calcium levels.

### CRAC Channels and Immune Responses

CRAC channels are located in the plasma membrane and open in response to the release of Ca2+ from endoplasmic reticulum stores. In immune cells, stimulation of cell surface receptors activates CRAC channels, leading to Ca2+ entry and cytokine production. Cells of both the adaptive and innate immune system (e.g., T-cells, neutrophils and macrophages) are known to be regulated by CRAC channels. CRAC channels also play a role in the activation of endothelial cells, which are involved in the pathogenesis of AKI.

Stimulation of T cell receptors causes depletion of intracellular Ca2+ stores and subsequent opening of the CRAC (Ca2+-release-activated Ca2+) channels. A sustained increase in intracellular Ca2+ concentration activates the calcineurin/NFAT (nuclear factor of activated T cells) pathway and turns on transcriptional programs of various cytokines. Orai1 and STIM1 are identified as a long-sought pore component of CRAC channels and as an endoplasmic reticulum (ER) Ca2+ sensor, respectively. STIM1 senses Ca2+ depletion in ER after stimulation of T cell receptors, translocates to plasma membrane (PM) proximal ER, binds to and activates Orai1. Human patients deficient in Orai1 or STIM1 have severe combined immune deficiency.

### Calcium Channel Inhibitors

Disclosed herein are a number of Calcium channel inhibitors consistent with the methods, compositions, administration regimens and compositions for use disclosed herein. In some embodiments a Calcium channel inhibitor is a SOC inhibitor. In some embodiments the Calcium channel inhibitor is a CRAC inhibitor. In some embodiments, the Calcium channel inhibitor inhibits a channel comprising STIM1 protein. In some embodiments, the Calcium channel inhibitor inhibits a channel comprising Orai1 protein. In some embodiments, the Calcium channel inhibitor inhibits a channel comprising Orai2 protein.

In some embodiments the compound is a compound having the structure of:

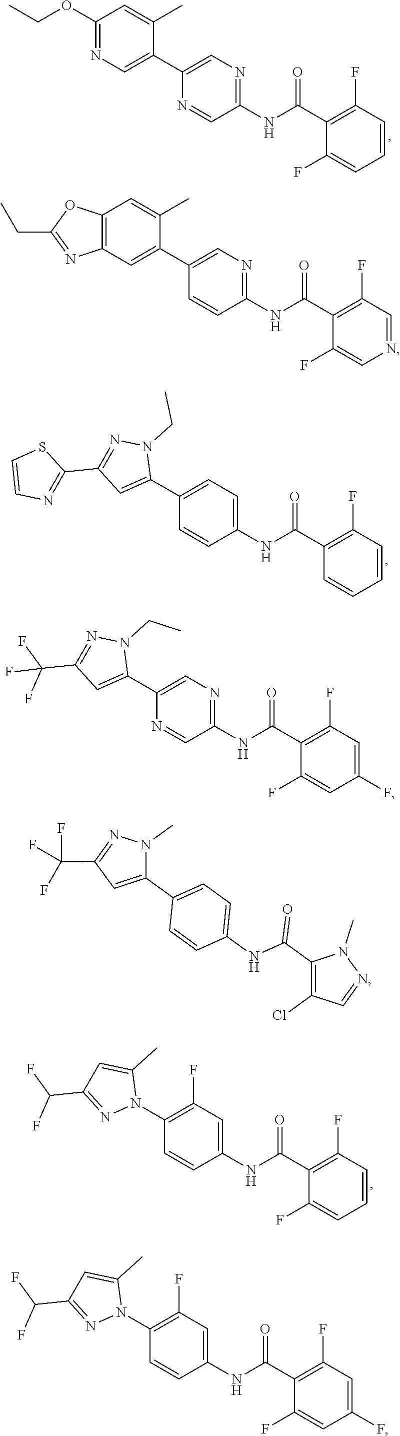

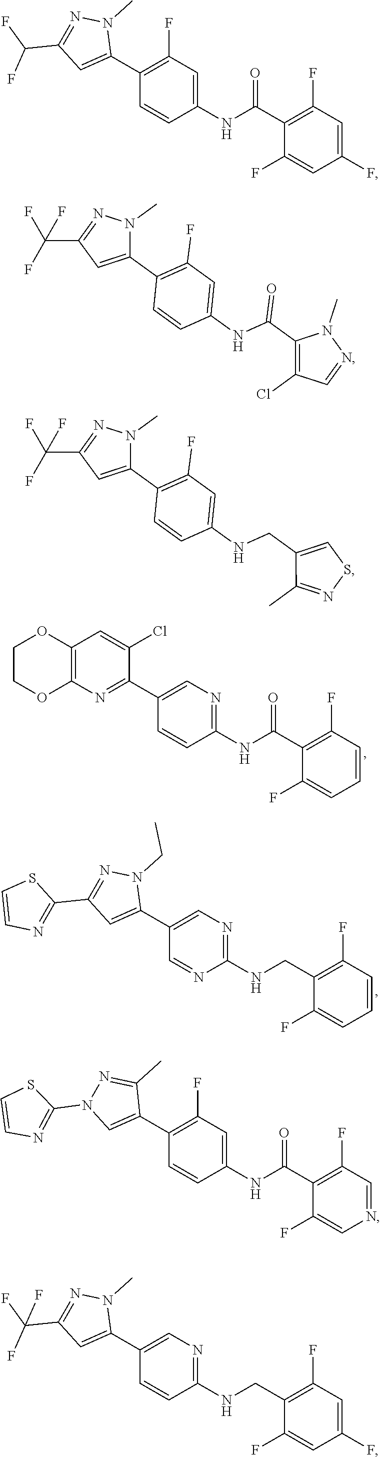

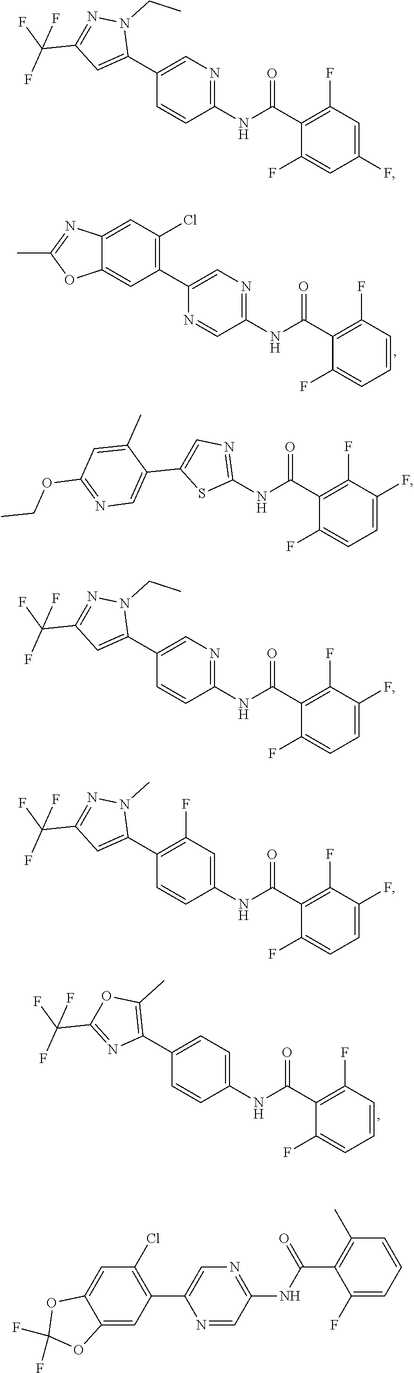

or a pharmaceutically acceptable salt, pharmaceutically acceptable solvate, or pharmaceutically acceptable prodrug thereof. In some embodiments the compound is selected form a list of compounds consisting: N-(5-(6-chloro-2,2-difluorobenzo[d][1,3]dioxol-5-yl)pyrazin-2-yl)-2-fluoro-6-methylbenzamide. In some aspects the intracellular Calcium signaling inhibitor is a compound of N-(5-(6-chloro-2,2-difluorobenzo[d][1,3]dioxol-5-yl)pyrazin-2-yl)-2-fluoro-6-methylbenzamide or a pharmaceutically acceptable salt, pharmaceutically acceptable solvate, or pharmaceutically acceptable prodrug thereof. In some aspects the intracellular Calcium signaling inhibitor is chosen from among the compounds, N-(5-(6-ethoxy-4-methylpyridin-3-yl)pyrazin-2-yl)-2,6-difluorobenzamide, N-(5-(2-ethyl-6-methylbenzo[d]oxazol-5-yl)pyridin-2-yl)-3,5-difluoroisonicotinamide, N-(4-(1-ethyl-3-(thiazol-2-yl)-1H-pyrazol-5-yl)phenyl)-2-fluorobenzamide, N-(5-(1-ethyl-3-(triflouromethyl)-1H-pyrazol-5-yl)pyrazin-2-yl)-2,4,6-trifluorobenzamide, 4-chloro-1-methyl-N-(4-(1-methyl-3-(trifluoromethyl)-1H-pyrazol-5-yl)phenyl)-1H-pyrazole-5-carboxamide, N-(4-(3-(difluoromethyl)-5-methyl-1H-pyrazol-1-yl)-3-fluorophenyl)-2,6-difluorobenzamide, N-(4-(3-(difluoromethyl)-5-methyl-1H-pyrazol-1-yl)-3-fluorophenyl)-2,4,6-trifluorobenzamide, N-(4-(3-(difluoromethyl)-1-methyl-1H-pyrazol-5-yl)-3-fluorophenyl)-2,4,6-trifluorobenzamide, 4-chloro-N-(3-fluoro-4-(1-methyl-3-(trifluoromethyl)-1H-pyrazol-5-yl)phenyl)-1-methyl-1H-pyrazole-5-carboxamide, 3-fluoro-4-(1-methyl-3-(trifluoromethyl)-1H-pyrazol-5-yl)-N-((3-methylisothiazol-4-yl)methyl)aniline, N-(5-(7-chloro-2,3-dihydro-[1,4]dioxino[2,3-b]pyridin-6-yl)pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl)isonicotinamide, 5-(1-methyl-3-(trifluoromethyl)-1H-pyrazol-5-yl)-N-(2,4,6-trifluorobenzyl)pyridin-2-amine, N-(5-(1-ethyl-3-(trifluoromethyl)-1H-pyrazol-5-yl)pyridin-2-yl)-2,4,6-trifluorobenzamide, N-(5-(5-chloro-2-methylbenzo[d]oxazol-6-yl)pyrazin-2-yl)-2,6-difluorobenzamide, N-(5-(6-ethoxy-4-methylpyridin-3-yl)thiazol-2-yl)-2,3,6-trifluorobenzamide, N-(5-(1-ethyl-3-(trifluoromethyl)-1H-pyrazol-5-yl)pyridin-2-yl)-2,3,6-trifluorobenzamide, 2,3,6-trifluoro-N-(3-fluoro-4-(1-methyl-3-(trifluoromethyl)-1H-pyrazol-5-yl)phenyl)benzamide, 2,6-difluoro-N-(4-(5-methyl-2-(trifluoromethyl)oxazol-4-yl)phenyl)benzamide, or N-(5-(6-chloro-2,2-difluorobenzo[d][1,3]dioxol-5-yl)pyrazin-2-yl)-2-fluoro-6-methylbenzamide or a pharmaceutically acceptable salt, pharmaceutically acceptable solvate, or pharmaceutically acceptable prodrug thereof.

### Further Forms of Compounds

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by the forming diastereomeric and separation by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

In some situations, compounds may exist as tautomers. All tautomers are included within the formulas described herein.

The methods and compositions described herein include the use of amorphous forms as well as crystalline forms (also known as polymorphs). The compounds described herein may be in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In some embodiments, compounds described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

To produce a prodrug, a pharmaceutically active compound is modified such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. In some embodiments, by virtue of knowledge of pharmacodynamic processes and drug metabolism in vivo, once a pharmaceutically active compound is determined, prodrugs of the compound are designed. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Saulnier et al., (1994), Bioorganic and Medicinal Chemistry Letters, Vol. 4, p. 1985; Rooseboom et al., Pharmacological Reviews, 56:53-102, 2004; Miller et al., J. Med. Chem. Vol. 46, no. 24, 5097-5116, 2003; Aesop Cho, “Recent Advances in Oral Prodrug Discovery”, Annual Reports in Medicinal Chemistry, Vol. 41, 395-407, 2006).

Prodrug forms of the herein described compounds, wherein the prodrug is metabolized in vivo to produce a compound as set forth herein, are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. Prodrugs may be designed as reversible drug derivatives, for use as modifiers to enhance drug transport to site-specific tissues. In some embodiments, the design of a prodrug increases the effective water solubility. See, e.g., Fedorak et al., Am. J. Physiol., 269: G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure).

Sites on the aromatic ring portion of compounds described herein can be susceptible to various metabolic reactions, therefore incorporation of appropriate substituents on the aromatic ring structures, such as, by way of example only, halogens can reduce, minimize or eliminate this metabolic pathway.

The compounds described herein may be labeled isotopically (e.g. with a radioisotope) or by other means, including, but not limited to, the use of chromophores or fluorescent moieties, bioluminescent labels, photoactivatable or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, 2H, 3H, 13C, 14C, 15N, 180, 170, 35S, 18F, 36Cl, respectively. Certain isotopically-labeled compounds described herein, for example those into which radioactive isotopes such as 3H and 14C are incorporated, are useful in drug and/or substrate tissue distribution assays. Further, substitution with isotopes such as deuterium, i.e., 2K, can afford certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In some embodiments, compounds described herein, are in various forms, including but not limited to, amorphous forms, milled forms, injectable emulsion forms, and nano-particulate forms. In addition, compounds described herein include crystalline forms, also known as polymorphs. Polymorphs include the different crystal packing arrangements of the same elemental composition of a compound. Polymorphs usually have different X-ray diffraction patterns, melting points, density, hardness, crystal shape, optical properties, stability, and solubility. Various factors such as the recrystallization solvent, rate of crystallization, and storage temperature may cause a single crystal form to dominate.

The screening and characterization of the pharmaceutically acceptable salts, polymorphs and/or solvates may be accomplished using a variety of techniques including, but not limited to, thermal analysis, x-ray diffraction, spectroscopy, vapor sorption, and microscopy. Thermal analysis methods address thermo chemical degradation or thermo physical processes including, but not limited to, polymorphic transitions, and such methods are used to analyze the relationships between polymorphic forms, determine weight loss, to find the glass transition temperature, or for excipient compatibility studies. Such methods include, but are not limited to, Differential scanning calorimetry (DSC), Modulated Differential Scanning Calorimetry (MDCS), Thermogravimetric analysis (TGA), and Thermogravi-metric and Infrared analysis (TG/IR). X-ray diffraction methods include, but are not limited to, single crystal and powder diffractometers and synchrotron sources. The various spectroscopic techniques used include, but are not limited to, Raman, FTIR, UV-VIS, and NMR (liquid and solid state). The various microscopy techniques include, but are not limited to, polarized light microscopy, Scanning Electron Microscopy (SEM) with Energy Dispersive X-Ray Analysis (EDX), Environmental Scanning Electron Microscopy with EDX (in gas or water vapor atmosphere), IR microscopy, and Raman microscopy.

Throughout the specification, groups and substituents thereof can be chosen to provide stable moieties and compounds.

### Synthesis of Compounds

In some embodiments, the synthesis of compounds described herein are accomplished using means described in the chemical literature, using the methods described herein, or by a combination thereof. In addition, solvents, temperatures and other reaction conditions presented herein may vary.

In other embodiments, the starting materials and reagents used for the synthesis of the compounds described herein are synthesized or are obtained from commercial sources, such as, but not limited to, Sigma-Aldrich, FischerScientific (Fischer Chemicals), and AcrosOrganics.

In further embodiments, the compounds described herein, and other related compounds having different substituents are synthesized using techniques and materials described herein as well as those that are recognized in the field, such as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4th Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999)(all of which are incorporated by reference for such disclosure).

### Acute Kidney Injury (AKI) and Inflammatory Responses

AKI is defined by an acute reduction in kidney function as identified by an increase in the serum creatinine and reduction in urine output. The severity of AKI is reflected by the AKI stage AKI 1-3, with stage 1 defined as a rise of serum creatinine level of >26 umol/L or 1.5 to 1.9 times the baseline serum creatinine; with stage 2 as a rise of serum creatinine level 2 to 2.9 times the baseline serum creatinine; with stage 3 as a rise of serum creatinine level 3 times the baseline serum creatinine or >354 umol/L.

The pathogenesis of AKI is complex. Renal ischemia/reperfusion (I/R) injury, one of the major causes of acute kidney injury (AKI), is associated with severe morbidity and mortality. Progression of chronic kidney disease (CKD) and end-stage kidney disease are recognized as possible outcomes for AKI patients. IR injury is caused by a reduction of renal blood flow below the limits of blood flow autoregulation. After the onset of reperfusion and lasting for a period of time, endothelial and epithelial cell injury may occur. Toxins may be another major factor that precipitate AKI. Although the initiating events of AKI may be different (e.g., sepsis, decreased blood volume, cardiac insufficiency), subsequent injury responses may involve similar signaling pathways.

More scientific evidence has suggested that inflammation/inflammatory responses may play a role in the pathogenesis of AKI. For example, IR injury may be associated with an inflammatory cascade and polymorphonuclear neutrophil (PMN) activation. Endothelial injury and dysfunction following renal ischemia has been shown to result in large releases of inflammatory mediators and adhesion molecules such as interleukin (IL)-1, IL-6, IL-8, IL-17, tumor necrosis factor (TNF)-α, P-selectin, E-selectin, intercellular adhesion molecule (ICAM)-1, etc. These cytokines induce tubular epithelial cell necrosis and renal tubular atrophy. Further, some studies have also demonstrated that the toll-like receptor (TLR4)/Nuclear factor-KB (NF-κB) pathway plays a dominant role in mediating deleterious effects in renal ischemia-reperfusion injury (IRI) by showing that TLR4 expressions increased in renal tubular epithelial cells after renal ischemia.

Other studies have shown that NACHT, LRR, and PYD domains-containing protein 3 (NLRP3) inflammasome plays a role in modulating kidney inflammation leading to several different renal disease models including I/R injury. The NLRP3 inflammasome is a cytoplasmic macromolecular complex that orchestrates early inflammatory responses of the innate immune system by inducingcaspase-1 activation and IL-10 maturation. Various danger signals, including mitochondrial reactive oxygen species (ROS), potassium efflux, and the release of lysosomal cathepsins, are identified as possible activators of the NLRP3 inflammasome. The necrotic tubular cells are capable of activating NLRP3 inflammasome in macrophages through the release of viable mitochondria. NLRP3-deficiency protects certain animal models, such as mice, against renal inflammation and tissue damage after I/R injury. In addition, NLRP3 is responsible for tubular apoptosis, whereas renal-associated NLRP3 impaired wound healing. The absence of NLRP3 in tubular cells improves regenerative response. These findings suggest that NLRP3 inflammasome could be a potential target for the treatment of renal I/R injury.

Moreover, other immune cell activities have been suggested to contribute renal injury or may enhance renal recovery. For example, renal CD4⁺ Th1 or Th17 cells are thought to exacerbate renal injury while T regulatory cells have been implicated in renal repair. Following recovery from I/R injury in rats, subsequent exposure to high-salt diet is shown to hasten the development of interstitial fibrosis, inflammation, proteinuria, and hypertension. These parameters of CKD progression are significantly attenuated by immunosuppression with mycophenolate, suggesting that lymphocyte activity also modulates the AKI-to-CKD transition. Naive CD4⁺ cells differentiate into effector T helper cells in the ischemic milieu, where they are exposed to different antigens and proinflammatory cytokines. T helper cells secrete various cytokines and are thought to orchestrate the adaptive immune response.

Th17 cells, which secrete the cytokine IL-17, are the prominent lymphocyte population found in rat kidney following I/R injury. These cells have been implicated in a variety of autoimmune diseases such as asthma, psoriasis, inflammatory bowel disease, and lupus erythematosus. Based on some studies, there is a significant expansion of Th17 cells in kidney within the first 3 days of I/R injury in rats, whereas Th17 levels resolve to near sham-operated control values within 7 days as renal function recovers. However, subsequent exposure of rats to high-salt diet (4%) strongly reactivates Th17 cell expression in post-ischemic kidney. This reactivation may contribute to CKD, since an IL-17R antagonist attenuated renal interstitial fibrosis and neutrophil infiltration in post I/R rats exposed to high-salt diet. Th17 cell differentiation is dependent on the activity of the transcription factor RORγT, and inhibitors of this factor can alleviate the pathological activation of Th17 cells. Activation of these cells by high-salt diet has also been demonstrated in a mouse model of autoimmune encephalitis and associated with the activity of serum and glucocorticoid regulated kinase (SGK-1) and nuclear factor of activated T cells 5 (NFAT5). Elevation of extracellular Na⁺ to 170 mM enhanced differentiation from naive CD4⁺ cells to Th17 cells in vitro in a process dependent on SGK-1. Th17 cell differentiation is dependent on the activity of the transcription factor RORγT and inhibitors of this factor can alleviate the pathological activation of Th17 cells. Activation of these cells by high salt diet has also been demonstrated in a mouse model of autoimmune encephalitis and associated with the activity of serum and glucocorticoid regulated kinase (SGK-1) and nuclear factor of activated T-cells 5 (NFAT5). Elevation of extracellular Na+ to 170 mM enhanced differentiation from naive CD4+ cells to Th17 cells in vitro in a process dependent SGK-1.

Previous studies have demonstrated that Orai1, the pore-forming subunit of Ca²⁺ release-activated Ca²⁺ channels (CRAC), is required for Th17 cell differentiation in vitro, partially due to NFAT activity. Orai1 mutant mice or inhibitors of Orai1 show impaired T cell receptor (TCR) activation and reduced IL-17 production, and are resistant to autoimmune disorders. Therefore, renal I/R may enhance lymphocyte Orai1-mediated Ca²⁺ signaling, which may drive Th17 cell expression and, in turn, modulates AKI and AKI-to-CKD progression. Ca²⁺ influx by Orai1 may be a mechanism that sustains the Th17-driven inflammatory response after AKI. In fact, some studies have shown that Orai1-expressing CD4⁺ T cells expand 48 hours after IR, which are restricted to IL-17-expressing cells. Orai1 expression remains elevated in post-AKI CD4⁺ T cells for up to a week, while Th17 response returns to baseline. Based on these observations, the sustained Orai1 expression in post-AKI CD4⁺ T cells may boost Th17 reactivation to a subsequent insult. Further, in vitro stimulation of post-AKI CD4⁺ T cells with angiotensin II (Ang II) and sodium (Na⁺) increase intracellular Ca²⁺, RORγT activity, and IL-17 (mRNA and protein) expression. These observations are substantiated by in vivo AKI-to-CKD studies in rats where high-salt administration after IR aggravated chronic renal inflammation, fibrosis, and impaired renal function.

Studies have also shown that Orai1 participates in AKI. For example, an expression level of a Ca2+ release-activated Ca2+ channel pore forming subunit OraM was measured in Th17 cells from kidneys obtained from renal injury mouse model. OraM was detected in Th17 cells and the number of these cells was increased following 1/R relative to sham mouse model. The total number CD4+/Orai1+ cells and the number of triple-positive CD4+/IL17+/Orai1+ cells in kidney were markedly elevated by 1/R injury. Studies have also shown that SOCE influences Th17 cells in AKI. For example, in AKI rats, SOCE inhibitors, such as YM58483/BPT2 attenuated the infiltration of total CD4+ T-cells, B-cells, and dendritic cells following 1/R. Total IL17 expressing cells were reduced in YM58483/BPT2 treated rats relative to vehicle treated rats. In addition, studies have shown that YM58483/BPT plays an important role in inhibition of Th17 cells in the early post-ischemic period in AKI.

More examples of SOCE inhibitors' influence on Th17 differentiation have been studies. One study showed that peripheral blood samples were obtained from critically ill patients with and without AKI. Samples were collected within 24-48 hours of AKI diagnosis for AKI cases or within 24-48 hours of ICU admission for frequency-matched (age, gender, baseline eGFR) controls without AKI. In isolated blood mononuclear cells, the percentages of total IL17+ cells and CD4+/IL17+ cells were significantly higher in AKI patients vs non-AKI patients. Moreover, the percentage of OraM positive cells was also prominently increased in non-AKI patients compared to patients with AKI. Similar to studies in rat kidney, Th17 cells were predominantly found within OraM expressing cells vs OraM-negative cells. Studies have demonstrated that the store-operated Ca²⁺ channel OraM is prominently induced in renal T-cells in the setting of kidney injury. Moreover, blockade of this channel attenuated Th17 cell induction and renal damage in response to ischemia/reperfusion injury. OraM mediated SOCE channel may be required for Th17 differentiation following 1/R, thus, OraM may represent a therapeutic target to attenuate AKI or immune mediated renal fibrosis and hypertension, which may occur secondary to AKI.

### Therapeutic Treatment of AKI

Disclosed herein are compositions and methods for treating acute kidney injury (AKI) in a subject comprising administering a therapeutically effective amount of an intracellular Calcium signaling inhibitor to said subject. Further, disclosed herein are compositions and methods for preventing AKI in a subject at risk of developing AKI, comprising administering a prophylactically effective amount of an intracellular Calcium signaling inhibitor to said subject. Moreover, disclosed herein are compositions and methods for preventing AKI from progressing to chronic kidney disease (CKD) in a subject comprising administering a prophylactically effective amount of an intracellular Calcium signaling inhibitor to said subject.

In some embodiments, the intracellular Calcium signaling inhibitor is delivered to achieve a tissue level concentration that is equal to, about, or greater than the in vitro IC₅₀ value determined for the compound. In some embodiments the Calcium signaling inhibitor is delivered to achieve a tissue level concentration that is 1.5×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or any non-integer multiple ranging from 1× to 100× of the in vitro IC₅₀ value determined for the compound.

In some embodiments the Calcium signaling inhibitor is delivered to achieve a tissue level concentration that ranges from 1× to 100×, 2× to 80×, 3× to 60×, 4× to 50×, 5× to 45×, 6× to 44×, 7× to 43×, 8× to 43×, 9× to 41×, or 10× to 40×, or any non-integer within said range, of the in vitro IC₅₀ value determined for the compound.

In some embodiments the Calcium signaling inhibitor is delivered to achieve a tissue level concentration that is 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61 μM, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM, 87 μM, 88 μM, 89 μM, 90 μM, 91 μM, 92 μM, 93 μM, 94 μM, 95 μM, 96 μM, 97 μM, 98 μM, 99 μM, 100 μM, or any non-integer multiple ranging from about 1 μM to about 100 μM.

In some embodiments the Calcium signaling inhibitor is delivered to achieve a tissue level concentration that ranges from 1 μM to 100 μM, 2 μM to 90 μM, 3 μM to 80 μM, 4 μM to 70 μM, 5 μM to 60 μM, 6 μM to 50 μM, 7 μM to 40 μM, 8 μM to 30 μM, 9 μM to 20 μM, or 10 μM to 40 μM, or any integer or non-integer within said range.

In some embodiments the Calcium signaling inhibitor is delivered to achieve a tissue level concentration that ranges from 9.5 μM to 10.5 μM, 9 μM to 11 μM, 8 μM to 12 μM, 7 μM to 13 μM, 5 μM to 15 μM, 2 μM to 20 μM or 1 μM to 50 μM, or any integer or non-integer within said range.

In some embodiments, the disclosed compound CM4620 in a suitable delivery method is able to inhibit differentiation of a CD4+ T cell to a T-helper 17 (TH17) cell. The circulating Th17 cells in a subject's blood following treatment is significantly reduced compared to prior to receiving the treatment. Further, following treatment, the percentages of total IL17+ cells and CD4+/IL17+ cells are reduced compared to prior to administration of CM4620. In addition, mRNA expression level and protein expression level of IL-17 are both decreased compared to prior to receiving CM4620.

The present disclosure also provides a method to decrease an amount of a Ca²⁺ release-activated Ca²⁺ channel pore forming subunit OraM, the method comprising administering to a mammal an effective amount of a Ca2+ release-activated (CRAC) channel inhibitor or a pharmaceutically acceptable salt thereof. In some embodiments, the CRAC channel inhibitor is CM4620.

Combination Administration with a Compound for Treating AKI

Disclosed herein are compositions and administration regimens for the combinatorial administration of a Calcium channel inhibitor and at least a compound for treating AKI. In some embodiments an administration regimen comprises administration to a subject of a compound for treating AKI, and administration of an intracellular Calcium signaling inhibitor.

In some embodiments, the compound is selected from the list consisting of a recombinant human IGF-I (rhIGF-I), atrial natriuretic peptide (ANP), dopamine, caspase inhibitor, minocycline, guanosine and Pifithrin-α(p53 Inhibitor), poly ADP-ribose polymerase inhibitor, deferoxamine, ethyl pyruvate, activated protein C, insulin, recombinant erythropoietin, hepatocyte growth factor, carbon monoxide release compound, bilirubin, endothelin antagonist, sphingosine 1 phosphate analog, adenosine analog, inducible nitric oxide synthase inhibitor, fibrate, neutrophil gelatinase-associated lipocalin, IL-6 antagonist, C5a antagonist, IL-10, dexmedetomidine, chloroquine (CQ), hydroxychloroquine (HCQ), and α-melanocyte-stimulating hormone.

### Various Compounds for Treating AKI

Antiapoptosis/Necrosis Agents

Caspase Inhibitors.

Caspases are a family of proteases that are involved in the initiation and execution phase of apoptosis. Nonselective and selective caspase inhibitors are effective in attenuating renal injury in ischemia- or endotoxemia-induced AKI when administered before or at the time of injury. Pancaspase inhibitors are in early clinical trials, and early targets include hepatitis C and orthotopic liver transplantation.

Minocycline.

Minocyclines are second-generation tetracycline antibiotics with proven human safety data. Minocycline is known to have antiapoptotic and anti-inflammatory effects. When administered 36 hour before renal ischemia, minocycline reduced tubular cell apoptosis and mitochondrial release of cytochrome c, p53, and bax. Furthermore, minocycline reduced kidney inflammation and also microvascular permeability. Minocycline has been used in clinical trials for rheumatoid arthritis and is undergoing testing in phase I/II clinical trials for amyotrophic lateral sclerosis.

Guanosine and Pifithrin-α (p53 Inhibitor).

GTP salvage by exogenous administration of guanosine reduced renal tubular cell apoptosis, an effect that was associated with inhibition of p53 expression. Pifithrin-α, a novel p53 inhibitor, also led to decreased tubule cell apoptosis and preserved renal function. This agent is nearing clinical trials in cancer therapy.

Poly ADP-Ribose Polymerase Inhibitor.

Poly ADP-ribose polymerase (PARP) is a ubiquitous nuclear enzyme that participates in DNA repair. Paradoxically, excessive activation of PARP from cellular injury leads to intracellular NAD⁺ and to ATP depletion, ultimately resulting in cell death. PARP overactivation has been known to play a role in the pathogenesis of IRI to kidney, heart, and brain. Inhibition of PARP immediately at reperfusion reduced injury. PARP inhibitors are in clinical trials for breast cancer (phase I) and cardiac reperfusion injury (phase II).

Free Radical Scavengers

Deferoxamine.

A key early feature of AKI is the generation of reactive oxygen species. The iron chelator deferoxamine is a widely known free radical scavenger. In several models of AKI, deferoxamine is proved effective. The protective effect of deferoxamine in various models suggests the central role of free radicals in AKI. Studies in AKI are planned to test the efficacy of iron chelation.

Antisepsis

Ethyl Pyruvate.

Pyruvate has been known as a potent endogenous antioxidant and free radical scavenger, and its derivative, ethyl pyruvate, proved to be effective in reducing mortality in animal models of lethal hemorrhagic shock and systemic inflammation caused by endotoxemia or sepsis. In addition to an effect on mortality, ethyl pyruvate reduced kidney injury using the technique cecal ligation puncture as a model of sepsis. Ethyl pyruvate is a widely used food additive and has been shown to be safe in phase I clinical trials. It now is being tested in a phase II trial in patients who undergo cardiopulmonary bypass surgery.

Activated Protein C

Activated protein C (APC) is a physiologic anticoagulant that is generated by thrombin-thrombomodulin complex in endothelial cells. In addition to its effect on coagulation, APC has been shown to have anti-inflammatory, antiapoptotic effects. APC also attenuated renal IRI by inhibiting leukocyte activation. APC is approved by the Food and Drug Administration for treating patients who have severe sepsis and an Acute Physiology, Age, Chronic Health Evaluation (APACHE) score of 25 or higher.

Insulin.

Insulin resistance and hyperglycemia are common in critically ill patients, and intensive insulin therapy that targeted blood glucose level between 80 and 110 mg/dl reduced the incidence of AKI that required dialysis or hemofiltration. The relationship of hyperglycemia and adverse outcome in critically ill patients with AKI also was observed recently in a study. The mechanism for clinical benefit may relate to the dosage of insulin as opposed to glycemic control. Endothelial dysfunction and subsequent hypercoagulation and dyslipidemia, commonly observed in critically ill patients, are corrected partially by insulin independent of its blood glucose-lowering effect.

Growth Factors

Recombinant Erythropoietin.

Erythropoietin has been shown to have anti-inflammatory and antiapoptotic effects in ischemic brain damage, spinal cord injury, and retinal damage. Exogenously administered erythropoietin before or at the time of reperfusion reduces kidney injury by reducing tubular necrosis and apoptosis. It enhanced tubular proliferation in cisplatin-induced AKI and also mediated mobilization and proliferation of endothelial progenitor cells from the bone marrow that has been shown to participate in tissue repair. Clinical use of recombinant erythropoietin should facilitate translation to human PKI.

Hepatocyte Growth Factor.

Hepatocyte growth factor (HGF) can promote cell growth, motility, and morphogenesis of various types of cells. Renal expression of HGF and its receptor, c-met, increases after IRI, and exogenous administration of HGF reduces renal injury and accelerates renal regeneration in a murine model of AKI. The mechanism of protection is thought to involve a decrease in leukocyte-endothelial interaction with reduced inflammation and also a decrease in tubular cell apoptosis. Currently, phase I/II study of recombinant human HGF in fulminant hepatic failure patients and another phase II study of HGF via plasmid vector in patients with critical limb ischemia and peripheral ischemic ulcer are underway. Experience in these clinical trials may shed light on human AKI.

Vasodilators

Carbon Monoxide Release Compounds and Bilirubin

In a seminal study, the heme oxygenase (HO) induction played a central role in limiting the extent of myoglobin-induced AKI. HO activity leads to the production of carbon monoxide (CO) and a potent antioxidant, bilirubin, and it is thought that the protective effect of HO activation is through these factors. In renal IRI administration of CO donor compounds tricarbonyldichlororuthenium (II) dimer ([Ru(CO)₃Cl₂]₂) or tricarbonylchloro(glycinato) ruthenium (II)([Ru(CO)₃Cl(glycinate)] 1 h before the onset of ischemia significantly decreased the levels of plasma creatinine 24 h after reperfusion as compared with vehicle-treated mice. This suggests that CO itself may be protective and limit renal damage in ischemia-induced AKI. Bilirubin also has been shown to reduce kidney injury from IRI, and when biliverdin and CO are used in combination, they are synergistic in improving heart allograft survival.

Endothelin Antagonist.

A potent vasoconstrictor, endothelin-1 (ET-1), has been implicated to play important roles in animal models of AKI or radiocontrast nephropathy. ET-1 mediates its biologic effects by binding to ET_{A} or ET_{B} receptors. In rat kidney. ET_{A} receptor stimulation is known to mediate vasoconstriction, whereas ET_{B} receptor activation also can mediate vasodilation by generation of nitric oxide and prostacyclin. In addition, ET-1 can stimulate the expression of adhesion molecules and the production of cytokines from monocytes and neutrophils, suggesting the possible role of ET-1 in inflammation in AKI. Several studies demonstrate the beneficial effect of selective ET_{A} or nonselective endothelin receptor antagonist in ischemic AKI, but the major limitation of those studies is that endothelin receptor antagonist was administered before injury. Tezosertan, a dual ET-1 receptor antagonist, attenuated renal injury even when administered after ischemia.

Sphingosine 1 Phosphate Analogs.

Sphingosine 1 phosphate (S1P) is a specific ligand for a family of G protein-coupled endothelial differentiation gene receptors (S1PR 1 through 5) that evoke diverse cellular signaling responses. S1PR regulate different biologic processes depending on their pattern of expression and the diverse G proteins present. S1P binds to receptors or acts as a second messenger to stimulate cell survival, inhibit cell apoptosis, and inhibit cell adhesion and movement. An S1P analog, FTY720, acts as an agonist at four S1PR, which lead to sequestration of lymphocytes in secondary lymphatic tissue. In studies of kidney IRI, FTY720 or similar compounds produced lymphopenia and renal tissue protection.

A_{2A} Agonists and Other Adenosine Analogs.

Adenosine binds to receptors, which are members of the G protein-coupled receptor family that includes four subtypes: A₁, A_{2A}, A_{2B}, and A₃Rs. Selective activation of A_{2A}Rs reduces parenchymal injury in nonrenal tissue, including heart, liver, spinal cord, lung, and brain. The selective A_{2A}R agonist ATL146e is highly protective against IRI of kidney and reduces injury by 70 to 80%. After administration either before or immediately at the onset of reperfusion, ATL146e alone or in combination with a phosphodiesterase inhibitor reduced renal injury. ATL146e is in human clinical studies for cardiac imaging, and current efforts are directed toward human clinical studies in AKI. Additional studies demonstrate that strategies that use A₁ agonists or A₃ blockers may be effective in AKI.

Inducible Nitric Oxide Synthase Inhibitors.

The role of nitric oxide (NO) and nitric oxide synthases (NOS) has been studied extensively. Both in vivo and in vitro studies point toward the important role of inducible NOS in mediating injury to proximal tubules.

Fibrates.

Peroxisome proliferator-activated receptors (PPAR) are transcription factors that regulate glucose and lipid metabolism. Recent studies indicated that PPAR play an important role in inflammation and immunity. Pretreatment of animals with fibrates (PPAR-α ligand) ameliorated cisplatin-induced renal dysfunction, and this was accompanied by suppression of NF-κB activation, cytokine/chemokine expression, and neutrophil infiltration, suggesting that the protective effect of fibrates is mediated through its anti-inflammatory effect.

In some embodiments the intracellular Calcium signaling inhibitor is an SOC inhibitor. In some embodiments the intracellular Calcium signaling inhibitor is a CRAC inhibitor. An exemplary CRAC inhibitor comprises N-(5-(6-Chloro-2,2-difluorobenzo[d][1,3]dioxol-5-yl)pyrazin-2-yl)-2-fluoro-6-methylbenzamide, having a structure of

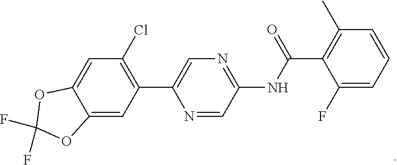 [SMILES: Cc1cccc(F)c1C(=O)Nc1cnc(-c2cc3c(cc2Cl)OC(F)(F)O3)cn1]

An exemplary CRAC inhibitor comprises GSK-7975A. An exemplary CRAC inhibitor comprises YM58483/BTP2. An exemplary CRAC inhibitor comprises 2,6-Difluoro-N-(1-(4-hydroxy-2-(trifluoromethyl)benzyl)-1H-pyrazol-3-yl)benzamide.

In some embodiments the administration regimen comprises administration of a calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2, and a compound for treating AKI. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered on the same day as a compound for treating AKI on lung activities. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered on the same week as a compound for treating AKI. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered concurrently with each administration of a compound for treating AKI. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered on an administration regimen pattern that is independent of the administration pattern for a compound for treating AKI. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered through the same route of delivery, such as orally or intravenously, as a compound for treating AKI. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered through a separate route of delivery compared to a compound for treating AKI. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered to a person receiving a compound for treating AKI only after said person shows at least one sign of an impact of said drug on lung activity. In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered to a person receiving a compound for treating AKI in the absence of any evidence in or from said person related to any sign of an impact of said compound on lung activity.

In some embodiments the calcium channel inhibitor such as a CRAC inhibitor such as at least one of N-(5-(6-Chloro-2,2-difluorobenzo[d][1,3]dioxol-5-yl)pyrazin-2-yl)-2-fluoro-6-methylbenzamide and BTP2 is administered in a single composition with a compound for treating AKI. Accordingly, some embodiments disclosed herein relate to a composition comprising an intracellular Calcium signaling inhibitor and at least one compound for treating AKI. In some embodiments the at least one drug selected from the list consisting of: a prostaglandin inhibitor, complement inhibitor, p-agonist, beta-2 agonist, granulocyte macrophage colony-stimulating factor, corticosteroid, N-acetylcysteine, statin, glucagon-like peptide-1 (7-36) amide (GLP-1), triggering receptor expressed on myeloid cells (TREM1) blocking peptide, 17-allylamino-17-demethoxygeldanamycin (17-AAG), antibody to tumor necrosis factor (TNF), recombinant interleukin (IL)-1 receptor antagonist, cisatracurium besilate, and Angiotensin-Converting Enzyme (ACE) Inhibitor.

In some embodiments, the intracellular Calcium signaling inhibitor is delivered to achieve a tissue level concentration that is equal to, about, or greater than the in vitro IC₅₀ value determined for the compound. In some embodiments the Calcium signaling inhibitor is delivered to achieve a tissue level concentration that is 1.5×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, or any non-integer multiple ranging from 1× to 100× of the in vitro IC₅₀ value determined for the compound.

In some embodiments the Calcium signaling inhibitor is delivered to achieve a tissue level concentration that ranges from 1× to 100×, 2× to 80×, 3× to 60×, 4× to 50×, 5× to 45×, 6× to 44×, 7× to 43×, 8× to 43×, 9× to 41×, or 10× to 40×, or any non-integer within said range, of the in vitro IC₅₀ value determined for the compound.

In some embodiments the Calcium signaling inhibitor is delivered to achieve a tissue level concentration that is 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35M, 36 μM, 37 μM, 38 μM, 39 μM, 40 μM, 41 μM, 42 μM, 43 μM, 44 μM, 45 μM, 46 μM, 47 μM, 48 μM, 49 μM, 50 μM, 51 μM, 52 μM, 53 μM, 54 μM, 55 μM, 56 μM, 57 μM, 58 μM, 59 μM, 60 μM, 61M, 62 μM, 63 μM, 64 μM, 65 μM, 66 μM, 67 μM, 68 μM, 69 μM, 70 μM, 71 μM, 72 μM, 73 μM, 74 μM, 75 μM, 76 μM, 77 μM, 78 μM, 79 μM, 80 μM, 81 μM, 82 μM, 83 μM, 84 μM, 85 μM, 86 μM 87 μM, 88 μM, 89 μM, 90 μM, 91 μM, 92 μM, 93 μM, 94 μM, 95 μM, 96 μM, 97 μM, 98 μM, 99 μM, 100 μM, or any non-integer multiple ranging from about 1 μM to about 100 μM.

In some embodiments the Calcium signaling inhibitor is delivered to achieve a tissue level concentration that ranges from 1 μM to 100 μM, 2 μM to 90 μM, 3 μM to 80 μM, 4 μM to 70 μM, 5 μM to 60 μM, 6 μM to 50 μM, 7 μM to 40 μM, 8 μM to 30 μM, 9 μM to 20 μM, or 10 μM to 40 μM, or any integer or non-integer within said range.

In some embodiments the Calcium signaling inhibitor is delivered to achieve a tissue level concentration that ranges from 9.5 μM to 10.5 μM, 9 μM to 11 μM, 8 μM to 12 μM, 7 μM to 13 μM, 5 μM to 15 μM, 2 μM to 20 μM or 1 μM to 50 μM, or any integer or non-integer within said range.

### Pharmaceutical Compositions

Provided herein can be pharmaceutical compositions comprising at least one of the Calcium signaling inhibitors described herein. In some cases, the pharmaceutical compositions comprise at least one of the Calcium signaling inhibitors and at least one of the compounds for treating AKI disclosed herein.

Pharmaceutical compositions provided herein can be introduced as oral forms, transdermal forms, oil formulations, edible foods, food substrates, aqueous dispersions, emulsions, injectable emulsions, solutions, suspensions, elixirs, gels, syrups, aerosols, mists, powders, capsule, tablets, nanoparticles, nanoparticle suspensions, nanoparticle emulsions, lozenges, lotions, pastes, formulated sticks, balms, creams, and/or ointments.

In some embodiments, the pharmaceutical composition additionally comprises at least one of an excipient, a solubilizer, a surfactant, a disintegrant, and a buffer. In some embodiments, the pharmaceutical composition is free of pharmaceutically acceptable excipients. The term “pharmaceutically acceptable excipient”, as used herein, means one or more compatible solid or encapsulating substances, which are suitable for administration to a subject. The term “compatible”, as used herein, means that the components of the composition are capable of being commingled with the subject compound, and with each other, in a manner such that there is no interaction, which would substantially reduce the pharmaceutical efficacy of the composition under ordinary use situations. In some embodiments, the pharmaceutically acceptable excipient is of sufficiently high purity and sufficiently low toxicity to render them suitable for administration preferably to an animal, preferably mammal, being treated.

Some examples of substances, which can serve as pharmaceutically acceptable excipients include: amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. In some embodiments, the amino acid is arginine. In some embodiments, the amino acid is L-arginine; monosaccharides such as glucose (dextrose), arabinose, mannitol, fructose (levulose), and galactose; cellulose and its derivatives such as sodium carboxymethyl cellulose, ethyl cellulose, and methyl cellulose; solid lubricants such as talc, stearic acid, magnesium stearate and sodium stearyl fumarate; polyols such as propyleneglycol, glycerin, sorbitol, mannitol, and polyethylene glycol; emulsifiers such as the polysorbates; wetting agents such as sodium lauryl sulfate, Tween®, Span, alkyl sulphates, and alkyl ethoxylate sulphates; cationic surfactants such as cetrimide, benzalkonium chloride, and cetylpyridinium chloride; diluents such as calcium carbonate, microcrystalline cellulose, calcium phosphate, starch, pregelatinized starch, sodium carbonate, mannitol, and lactose; binders such as starches (corn starch and potato starch), gelatin, sucrose hydroxypropyl cellulose (HPC), polyvinylpyrrolidone (PVP), and hydroxypropyl methyl cellulose (HPMC); disintegrants such as starch, and alginic acid; super-disintegrants such as ac-di-sol, croscarmellose sodium, sodium starch glycolate and crospovidone.

Glidants such as silicon dioxide; coloring agents such as the FD&C dyes; sweeteners and flavoring agents, such as aspartame, saccharin, menthol, peppermint, and fruit flavors; preservatives such as benzalkonium chloride, PHMB, chlorobutanol, thimerosal, phenylmercuric, acetate, phenylmercuric nitrate, parabens, and sodium benzoate; tonicity adjustors such as sodium chloride, potassium chloride, mannitol, and glycerin; antioxidants such as sodium bisulfite, acetone sodium bisulfite, sodium formaldehyde, sulfoxylate, thiourea, and EDTA; pH adjuster such as NaOH, sodium carbonate, sodium acetate, HCl, and citric acid; cryoprotectants such as sodium or potassium phosphates, citric acid, tartaric acid, gelatin, and carbohydrates such as dextrose, mannitol, and dextran; surfactants such as sodium lauryl sulfate. For example, cationic surfactants such as cetrimide (including tetradecyl trimethyl ammonium bromide with dodecyl and hexadecyl compounds), benzalkonium chloride, and cetylpyridinium chloride. Some examples of anionic surfactants are alkylsulphates, alkylethoxylate sulphates, soaps, carxylate ions, sulfate ions, and sulfonate ions. Some examples of non-ionic surfactants are polyoxyethylene derivatives, polyoxypropylene derivatives, polyol derivatives, polyol esters, polyoxyethylene esters, poloxamers, glocol, glycerol esters, sorbitan derivatives, polyethylene glycol (such as PEG-40, PEG-50, or PEG-55) and esters of fatty alcohols; organic materials such as carbohydrates, modified carbohydrates, lactose (including a-lactose, monohydrate spray dried lactose or anhydrous lactose), starch, pregelatinized starch, sucrose, mannitol, sorbital, cellulose (including powdered cellulose and microcrystalline cellulose); inorganic materials such as calcium phosphates (including anhydrous dibasic calcium hosphate, dibasic calcium phosphate or tribasic calcium phosphate); co-processed diluents; compression aids; anti-tacking agents such as silicon dioxide and talc.

In some embodiments, the pharmaceutical compositions described herein are provided in unit dosage form. As used herein, a “unit dosage form” is a composition containing an amount of the at least one of the Calcium signaling inhibitors and/or the at least one of the compounds for treating AKI that is suitable for administration to a subject in a single dose, according to good medical practice. The preparation of a single or unit dosage form however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded.

### Certain Terminology

Unless defined otherwise, all technical and scientific terms used herein have the same meaning as is commonly understood to which the claimed subject matter pertains. In the event that there is a plurality of definitions for terms herein, those in this section prevail. Where reference is made to a URL or other such identifier or address, it is understood that such identifiers can change and particular information on the internet can come and go, but equivalent information can be found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of“or” means “and/or” unless stated otherwise. Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms may be found in reference works, including but not limited to, Carey and Sundberg “Advanced Organic Chemistry 4th Ed.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology.

Unless specific definitions are provided, the nomenclature employed in connection with, and the laboratory procedures and techniques of, analytical chemistry, synthetic organic chemistry, and medicinal and pharmaceutical chemistry described herein are those recognized in the field. Standard techniques can be used for chemical syntheses, chemical analyses, pharmaceutical preparation, formulation, and delivery, and treatment of patients. Standard techniques can be used for recombinant DNA, oligonucleotide synthesis, and tissue culture and transformation (e.g., electroporation, lipofection). Reactions and purification techniques can be performed e.g., using kits of manufacturer's specifications or as commonly accomplished in the art or as described herein. The foregoing techniques and procedures can be generally performed of conventional methods and as described in various general and more specific references that are cited and discussed throughout the present specification.

It is to be understood that the methods and compositions described herein are not limited to the particular methodology, protocols, cell lines, constructs, and reagents described herein and as such may vary. It is also to be understood that the terminology used herein is for the purpose of describing particular embodiments only, and is not intended to limit the scope of the methods, compounds, compositions described herein.

The terms “kit” and “article of manufacture” are used as synonyms.

The term “subject” or “patient” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” as used herein, include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. As used herein, the term “target protein” refers to a protein or a portion of a protein capable of being bound by, or interacting with a compound described herein, such as a compound with a structure from the group of Compound A. In certain embodiments, a target protein is a STIM protein. In certain embodiments, a target protein is an Orai protein.

As used herein, “STIM protein” includes but is not limited to, mammalian STIM-1, such as human and rodent (e.g., mouse) STIM-1, Drosophila melanogaster D-STIM, C. elegans C-STIM, Anopheles gambiae STIM and mammalian STIM-2, such as human and rodent (e.g., mouse) STIM-2. (see paragraphs [0211] through [0270] of US 2007/0031814, as well as Table 3 of US 2007/0031814, herein incorporated by reference) As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum).

As used herein, an “Orai protein” includes Orai1 (SEQ ID NO: 1 as described in WO 07/081804), Orai2 (SEQ ID NO: 2 as described in WO 07/081804), or Orai3 (SEQ ID NO: 3 as described in WO 07/081804). Orai1 nucleic acid sequence corresponds to GenBank accession number NM_032790, Orai2 nucleic acid sequence corresponds to GenBank accession number BC069270 and Orai3 nucleic acid sequence corresponds to GenBank accession number NM_152288. As used herein, Orai refers to any one of the Orai genes, e.g., Orai1, Orai2, Orai3 (see Table I of WO 07/081804). As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum).

The term “fragment” or “derivative” when referring to a protein (e.g. STIM, Orai) means proteins or polypeptides which retain essentially the same biological function or activity in at least one assay as the native protein(s). For example, the fragments or derivatives of the referenced protein maintains at least about 50% of the activity of the native proteins, at least 75%, at least about 95% of the activity of the native proteins, as determined e.g. by a calcium influx assay.

As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that can be attributed to or associated with administration of the compound or composition.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target. For example, a modulator can cause an increase or decrease in the magnitude of a certain activity of a target compared to the magnitude of the activity in the absence of the modulator. In certain embodiments, a modulator is an inhibitor, which decreases the magnitude of one or more activities of a target. In certain embodiments, an inhibitor completely prevents one or more activities of a target.

As used herein, “modulation” with reference to intracellular calcium refers to any alteration or adjustment in intracellular calcium including but not limited to alteration of calcium concentration in the cytoplasm and/or intracellular calcium storage organelles, e.g., endoplasmic reticulum, and alteration of the kinetics of calcium fluxes into, out of and within cells. In aspect, modulation refers to reduction.

As used herein, the term “target activity” refers to a biological activity capable of being modulated by a modulator. Certain exemplary target activities include, but are not limited to, binding affinity, signal transduction, enzymatic activity, tumor growth, inflammation or inflammation-related processes, and amelioration of one or more symptoms associated with a disease or condition.

The terms “inhibit”, “inhibiting”, or “inhibitor” of SOC channel activity or CRAC channel activity, as used herein, refer to inhibition of store operated calcium channel activity or calcium release activated calcium channel activity.

The term “acceptable” with respect to a formulation, composition or ingredient, as used herein, means having no persistent detrimental effect on the general health of the subject being treated.

The term “pharmaceutically acceptable,” as used herein, refers a material, such as a carrier, diluent, or formulation, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutical combination” as used herein, means a product that results from the mixing or combining of more than one active ingredient and includes both fixed and non-fixed combinations of the active ingredients. The term “fixed combination” means that one active ingredient, e.g. a compound with a structure from the group of Compound A and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound with a structure from the group of Compound A, described herein with other chemical components, such as carriers, stabilizers, diluents, surfactants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, subcutaneous, intramuscular, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition that includes a compound with a structure from the group of Compound A, required to provide a clinically significant decrease in disease symptoms. An appropriate “effective” amount in any individual case may be determined using techniques, such as a dose escalation study.

In prophylactic applications, compositions described herein are administered to a subject susceptible to or otherwise at risk of a particular disease, disorder or condition, such as AKI to prevent the subject from developing AKI. Further, if a subject has already developed AKI, a prophylactic application of the disclosed compositions is to prevent the subject from progressing from AKI to chronic kidney disease (CKD). Such an amount is defined to be a “prophylactically effective amount or dose.” In this use, the precise amounts also depend on the subject's state of health, weight, and the like. When used in a subject, effective amounts for this use will depend on the severity and course of the disease, disorder or condition, previous therapy, the subject's health status and response to the drugs, and the judgment of the treating physician.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “co-administration” or the like, as used herein, are meant to encompass administration of the selected therapeutic agents to a single patient, and are intended to include treatment regimens in which the agents are administered by the same or different route of administration or at the same or different time.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds.

“Bioavailability” refers to the percentage of the weight of the compound disclosed herein (e.g. a compound from the group of Compound A) that is delivered into the general circulation of the animal or human being studied. The total exposure (AUC(0-∞)) of a drug when administered intravenously is usually defined as 100% bioavailable (F %). “Oral bioavailability” refers to the extent to which a compound disclosed herein, is absorbed into the general circulation when the pharmaceutical composition is taken orally as compared to intravenous injection.

“Blood plasma concentration” refers to the concentration of a compound with a structure from the group of Compound A, in the plasma component of blood of a subject. It is understood that the plasma concentration of compounds described herein may vary significantly between subjects, due to variability with respect to metabolism and/or possible interactions with other therapeutic agents. In accordance with one embodiment disclosed herein, the blood plasma concentration of the compounds disclosed herein may vary from subject to subject. Likewise, values such as maximum plasma concentration (Cmax) or time to reach maximum plasma concentration (Tmax), or total area under the plasma concentration time curve (AUC(0-∞)) may vary from subject to subject. Due to this variability, the amount necessary to constitute “a therapeutically effective amount” of a compound may vary from subject to subject.

As used herein, “calcium homeostasis” refers to the maintenance of an overall balance in intracellular calcium levels and movements, including calcium signaling, within a cell.

As used herein, “intracellular calcium” refers to calcium located in a cell without specification of a particular cellular location. In contrast, “cytosolic” or “cytoplasmic” with reference to calcium refers to calcium located in the cell cytoplasm.

As used herein, an effect on intracellular calcium is any alteration of any aspect of intracellular calcium, including but not limited to, an alteration in intracellular calcium levels and location and movement of calcium into, out of or within a cell or intracellular calcium store or organelle. For example, an effect on intracellular calcium can be an alteration of the properties, such as, for example, the kinetics, sensitivities, rate, amplitude, and electrophysiological characteristics, of calcium flux or movement that occurs in a cell or portion thereof. An effect on intracellular calcium can be an alteration in any intracellular calcium-modulating process, including, store-operated calcium entry, cytosolic calcium buffering, and calcium levels in or movement of calcium into, out of or within an intracellular calcium store. Any of these aspects can be assessed in a variety of ways including, but not limited to, evaluation of calcium or other ion (particularly cation) levels, movement of calcium or other ion (particularly cation), fluctuations in calcium or other ion (particularly cation) levels, kinetics of calcium or other ion (particularly cation) fluxes and/or transport of calcium or other ion (particularly cation) through a membrane. An alteration can be any such change that is statistically significant. Thus, for example if intracellular calcium in a test cell and a control cell is said to differ, such difference can be a statistically significant difference.

As used herein, “involved in” with respect to the relationship between a protein and an aspect of intracellular calcium or intracellular calcium regulation means that when expression or activity of the protein in a cell is reduced, altered or eliminated, there is a concomitant or associated reduction, alteration or elimination of one or more aspects of intracellular calcium or intracellular calcium regulation. Such an alteration or reduction in expression or activity can occur by virtue of an alteration of expression of a gene encoding the protein or by altering the levels of the protein. A protein involved in an aspect of intracellular calcium, such as, for example, store-operated calcium entry, thus, can be one that provides for or participates in an aspect of intracellular calcium or intracellular calcium regulation. For example, a protein that provides for store-operated calcium entry can be a STIM protein and/or an Orai protein.

As used herein, a protein that is a component of a calcium channel is a protein that participates in multi-protein complex that forms the channel.

As used herein, “basal” or “resting” with reference to cytosolic calcium levels refers to the concentration of calcium in the cytoplasm of a cell, such as, for example, an unstimulated cell, that has not been subjected to a condition that results in movement of calcium into or out of the cell or within the cell. The basal or resting cytosolic calcium level can be the concentration of free calcium (i.e., calcium that is not bound to a cellular calcium-binding substance) in the cytoplasm of a cell, such as, for example, an unstimulated cell, that has not been subjected to a condition that results in movement of calcium into or out of the cell.

As used herein, “movement” with respect to ions, including cations, e.g., calcium, refers to movement or relocation, such as for example flux, of ions into, out of, or within a cell. Thus, movement of ions can be, for example, movement of ions from the extracellular medium into a cell, from within a cell to the extracellular medium, from within an intracellular organelle or storage site to the cytosol, from the cytosol into an intracellular organelle or storage site, from one intracellular organelle or storage site to another intracellular organelle or storage site, from the extracellular medium into an intracellular organelle or storage site, from an intracellular organelle or storage site to the extracellular medium and from one location to another within the cell cytoplasm.

As used herein, “cation entry” or “calcium entry” into a cell refers to entry of cations, such as calcium, into an intracellular location, such as the cytoplasm of a cell or into the lumen of an intracellular organelle or storage site. Thus, cation entry can be, for example, the movement of cations into the cell cytoplasm from the extracellular medium or from an intracellular organelle or storage site, or the movement of cations into an intracellular organelle or storage site from the cytoplasm or extracellular medium. Movement of calcium into the cytoplasm from an intracellular organelle or storage site is also referred to as “calcium release” from the organelle or storage site.

As used herein, “protein that modulates intracellular calcium” refers to any cellular protein that is involved in regulating, controlling and/or altering intracellular calcium. For example, such a protein can be involved in altering or adjusting intracellular calcium in a number of ways, including, but not limited to, through the maintenance of resting or basal cytoplasmic calcium levels, or through involvement in a cellular response to a signal that is transmitted in a cell through a mechanism that includes a deviation in intracellular calcium from resting or basal states. In the context of a “protein that modulates intracellular calcium,” a “cellular” protein is one that is associated with a cell, such as, for example, a cytoplasmic protein, a plasma membrane-associated protein or an intracellular membrane protein. Proteins that modulate intracellular calcium include, but are not limited to, ion transport proteins, calcium-binding proteins and regulatory proteins that regulate ion transport proteins.

As used herein, “cell response” refers to any cellular response that results from ion movement into or out of a cell or within a cell. The cell response may be associated with any cellular activity that is dependent, at least in part, on ions such as, for example, calcium. Such activities may include, for example, cellular activation, gene expression, endocytosis, exocytosis, cellular trafficking and apoptotic cell death.

As used herein, “immune cells” include cells of the immune system and cells that perform a function or activity in an immune response, such as, but not limited to, T-cells, B-cells, lymphocytes, macrophages, dendritic cells, neutrophils, eosinophils, basophils, mast cells, plasma cells, white blood cells, antigen presenting cells and natural killer cells.

As used herein, “cytokine” refers to small soluble proteins secreted by cells that can alter the behavior or properties of the secreting cell or another cell. Cytokines bind to cytokine receptors and trigger a behavior or property within the cell, for example, cell proliferation, death or differentiation. Exemplary cytokines include, but are not limited to, interleukins (e.g., IL-2, IL-3, IL-4, IL-5, IL-6, IL-7, IL-8, IL-9, IL-10, IL-I 1, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF.

“Store operated calcium entry” or “SOCE” refers to the mechanism by which release of calcium ions from intracellular stores is coordinated with ion influx across the plasma membrane.

“Selective inhibitor of SOC channel activity” means that the inhibitor is selective for SOC channels and does not substantially affect the activity of other types of ion channels.

“Selective inhibitor of CRAC channel activity” means that the inhibitor is selective for CRAC channels and does not substantially affect the activity of other types of ion channels and/or other SOC channels.

As used herein, the term ‘calcium’ may be used to refer to the element or to the divalent cation Ca²⁺.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

Example 1: Phase 1 Clinical Trial. An open-label study is performed to evaluate the safety, tolerability, pharmacokinetics and pharmacodynamics of the pharmaceutical compositions disclosed herein on subjects having AKI or at risk for developing AKI, such as subjects having sepsis, hypovolaemia, and diabetes, that are likely to lead to complications such as AKI during hospitalization.

Single ascending dose (SAD) arms: subjects in each group receive either a single dose of the pharmaceutical composition or a placebo. Exemplary doses are 0.1, 0.5, 1, 2, 3, 4, 5, 10, 20, 25, 30, 35, 40, 45, 50, 55, 60, 65, 70, 75, 80, 85, 90, 95, 100 mg of the pharmaceutical composition per kg of the subject's weight. Safety monitoring and PK assessments are performed for a predetermined time. Based on evaluation of the PK data, and if the pharmaceutical composition is deemed to be well tolerated, dose escalation occurs, either within the same groups or a further group of healthy subjects. Dose escalation continues until the maximum dose has been attained unless predefined maximum exposure is reached or intolerable side effects become apparent.

Multiple ascending dose (MAD) arms: Subjects in each group receive multiple doses of the pharmaceutical composition or a placebo. The dose levels and dosing intervals are selected as those that are predicted to be safe from the SAD data. Dose levels and dosing frequency are chosen to achieve therapeutic drug levels within the systemic circulation that are maintained at steady state for several days to allow appropriate safety parameters to be monitored. Samples are collected and analyzed to determination PK profiles.

Outcome measures include determining a test subject's serum creatinine level over baseline level 12, 24, 48, 72 hours, 1 week, 2 weeks, 3 weeks, 4 weeks, 6 weeks, 8 weeks after receiving intravenous injections of the pharmaceutical composition disclosed herein. Estimated glomerular filtration rates (eGFRs) of the test subject are also measured after 12, 24, 48, 72 hours, 1 week, 2 weeks, 3 weeks, 4 weeks, 6 weeks, 8 weeks after receiving intravenous injections of the pharmaceutical composition disclosed herein. eGFR is equal to the total of the filtration rates of the functioning nephrons in the kidney. GFR is considered the optimal way to measure kidney function, which in conjunction with albuminuria, can help determine the extent of CKD in an individual. A rise in blood creatinine levels is observed only after significant loss of functioning nephrons. The standard for measuring GFR is using plasma or urinary clearance of an exogenous filtration marker. However, this is a complex procedure and generally not routinely performed. Therefore, GFR is usually estimated from the subject's serum creatinine and/or cystatin C level, in combination with demographic factors such as age, race, and gender using an estimating equation. Serum urea levels and inulin clearance may also be used to estimate GFR of a subject.

Patient Exclusion Criteria: Patients with a history of dialysis (hemodialysis, peritoneal dialysis), under the age of 18, or no evidence of pre-existing CKD will be excluded.

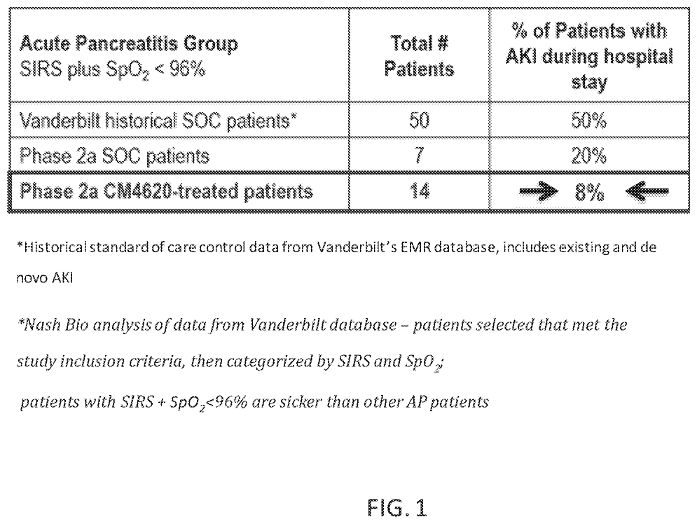

Example 2: evaluation of existing and/or de novo AKI in patients with acute pancreatitis: a group of patients with acute pancreatitis was studied and evaluated to see an intracellular Calcium signaling inhibitor's effects in preventing AKI. The group of patients with acute pancreatitis were divided in two sub-groups, one sub-group received treatment of CM4620 injectable emulsion (CM4620-IE) and the other sub-group received control treatment without receiving CM4620-IE. As illustrated in FIG. 1, the sub-group received control treatment has 20% of the patients developing AKI, whereas, the sub-group received CM4620-IE treatment has only 8% of the patients developing AKI. Further, patients with acute pancreatitis that met the inclusion criteria (disclosed below) from Vanderbilt database was also evaluated and 50% of the patients developed AKI as shown in FIG. 1. These patients did not receive CM4620-IE as their treatment.

The Inclusion Criteria is listed as below

Diagnosis of acute pancreatitis established by the presence of abdominal pain consistent with acute pancreatitis, and 1 of the following 2 criteria:
Serum lipase and/or serum amylase>3 times the upper limit of normal (ULN);
Characteristic findings of acute pancreatitis on abdominal imaging;
Adults ≥18 years of age;
A female patient of child-bearing potential who is sexually active with a male partner must be willing to practice acceptable methods of birth control for 365 days after the last dose of CM4620-IE;
A male patient who is sexually active with a female partner of childbearing potential must be willing to practice acceptable methods of birth control for 365 days after the last dose of CM4620-IE and must not donate sperm for 365 days;
Willing and able to, or have a legal authorized representative (LAR) who is willing and able to, provide informed consent to participate, and cooperate with all aspects of the protocol.

The Exclusion Criteria is listed below:

Any concurrent clinical condition that a study physician believes could potentially pose an unacceptable health risk to the patient while involved in the study or may limit expected survival to <6 months;
Suspected presence of cholangitis in the judgment of the treating investigator;
Any malignancy being treated with chemotherapy or immunotherapy;
Any autoimmune disease being treated with immunosuppressive medication or immunotherapy;
History of: chronic pancreatitis, pancreatic necrosectomy, or pancreatic enzyme replacement therapy; Biopsy proven cirrhosis, portal hypertension, hepatic failure/hepatic encephalopathy;
Known hepatitis B or C, or HIV: History of organ or hematologic transplant; Myocardial infarction, revascularization, cardiovascular accident (CVA) in the 30 days prior to Day 1;
Current renal replacement therapy;
Current known abuse of cocaine or methamphetamine;
Known to be pregnant or are nursing;
Participated in another study of an investigational drug or therapeutic medical device in the 30 days prior to Day 1;
History of allergy to eggs or known hypersensitivity to any components of CM4620-IE; Prior treatment with CM4620-IE.

1. A method for treating acute kidney injury (AKI) in a subject comprising administering a therapeutically effective amount of an intracellular Calcium signaling inhibitor to said subject.

2. (canceled)

3. (canceled)

4. The method of claim 1, wherein said intracellular Calcium signaling inhibitor is a store-operated calcium (SOC) channel inhibitor.

5. The method of claim 1, wherein said intracellular Calcium signaling inhibitor is a Ca2+ release-activated (CRAC) channel inhibitor.

6. The method of claim 1, wherein said intracellular Calcium signaling inhibitor inhibits a channel comprising a stromal interaction molecule 1 (STIM1) protein.

7. The method of claim 1, wherein said intracellular Calcium signaling inhibitor inhibits a channel comprising Orai1 protein.

8. The method of claim 1, wherein said intracellular Calcium signaling inhibits a channel comprising Orai2 protein.

9. The method of claim 1, wherein said intracellular Calcium signaling inhibitor is a compound having the structure of: N-(5-(6-ethoxy-4-methylpyridin-3-yl)pyrazin-2-yl)-2,6-difluorobenzamide, N-(5-(2-ethyl-6-methylbenzo[d]oxazol-5-yl)pyridin-2-yl)-3,5-difluoroisonicotinamide, N-(4-(1-ethyl-3-(thiazol-2-yl)-1H-pyrazol-5-yl)phenyl)-2-fluorobenzamide, N-(5-(1-ethyl-3-(triflouromethyl)-1H-pyrazol-5-yl)pyrazin-2-yl)-2,4,6-trifluorobenzamide, 4-chloro-1-methyl-N-(4-(1-methyl-3-(trifluoromethyl)-1H-pyrazol-5-yl)phenyl)-1H-pyrazole-5-carboxamide, N-(4-(3-(difluoromethyl)-5-methyl-1H-pyrazol-1-yl)-3-fluorophenyl)-2,6-difluorobenzamide, N-(4-(3-(difluoromethyl)-5-methyl-1H-pyrazol-1-yl)-3-fluorophenyl)-2,4,6-trifluorobenzamide, N-(4-(3-(difluoromethyl)-1-methyl-1H-pyrazol-5-yl)-3-fluorophenyl)-2,4,6-trifluorobenzamide, 4-chloro-N-(3-fluoro-4-(1-methyl-3-(trifluoromethyl)-1H-pyrazol-5-yl)phenyl)-1-methyl-1H-pyrazole-5-carboxamide, 3-fluoro-4-(1-methyl-3-(trifluoromethyl)-1H-pyrazol-5-yl)-N-((3-methylisothiazol-4-yl)methyl)aniline, N-(5-(7-chloro-2,3-dihydro-[1,4]dioxino[2,3-b]pyridin-6-yl)pyridin-2-yl)-2,6-difluorobenzamide, N-(2,6-difluorobenzyl)-5-(1-ethyl-3-(thiazol-2-yl)-1H-pyrazol-5-yl)pyrimidin-2-amine, 3,5-difluoro-N-(3-fluoro-4-(3-methyl-1-(thiazol-2-yl)-1H-pyrazol-4-yl)phenyl)isonicotinamide, 5-(1-methyl-3-(trifluoromethyl)-1H-pyrazol-5-yl)-N-(2,4,6-trifluorobenzyl)pyridin-2-amine, N-(5-(1-ethyl-3-(trifluoromethyl)-1H-pyrazol-5-yl)pyridin-2-yl)-2,4,6-trifluorobenzamide, N-(5-(5-chloro-2-methylbenzo[d]oxazol-6-yl)pyrazin-2-yl)-2,6-difluorobenzamide, N-(5-(6-ethoxy-4-methylpyridin-3-yl)thiazol-2-yl)-2,3,6-trifluorobenzamide, N-(5-(1-ethyl-3-(trifluoromethyl)-1H-pyrazol-5-yl)pyridin-2-yl)-2,3,6-trifluorobenzamide, 2,3,6-trifluoro-N-(3-fluoro-4-(1-methyl-3-(trifluoromethyl)-1H-pyrazol-5-yl)phenyl)benzamide, 2,6-difluoro-N-(4-(5-methyl-2-(trifluoromethyl)oxazol-4-yl)phenyl)benzamide, or N-(5-(6-chloro-2,2-difluorobenzo[d][1,3]dioxol-5-yl)pyrazin-2-yl)-2-fluoro-6-methylbenzamide, or a pharmaceutically acceptable salt, pharmaceutically acceptable solvate, or pharmaceutically acceptable prodrug thereof.

10. The method of claim 9, wherein said intracellular Calcium signaling inhibitor is a compound of chemical name N-(5-(6-Chloro-2,2-difluorobenzo[d][1,3]dioxol-5-yl)pyrazin-2-yl)-2-fluoro-6-methylbenzamide or a pharmaceutically acceptable salt, pharmaceutically acceptable solvate, or pharmaceutically acceptable prodrug thereof.

11. The method of claim 9, wherein said intracellular Calcium signaling inhibitor is a compound of chemical name N-(2,6-difluorobenzyl)-5-(1-ethyl-3-thiazol-2-yl)-1H-pyrazol-5-yl)pyrimidin-2-amine or a pharmaceutically acceptable salt, pharmaceutically acceptable solvate, or pharmaceutically acceptable prodrug thereof.

12. The method of claim 1, further comprising inhibiting differentiation of a CD4+ T cell to a T-helper 17 (TH17) cell.

13. The method of claim 12, wherein the differentiation of a CD4+ T cell to a TH17 cell occurs in a kidney.

14. The method of claim 1, further comprising reducing an amount of pro-inflammatory cytokine Interleukin 17 (IL-17).

15. The method of claim 1, further comprising administering a second compound selected from the group consisting of a recombinant human IGF-I (rhIGF-I), atrial natriuretic peptide (ANP), dopamine, caspase inhibitor, minocycline, guanosine and Pifithrin-α (p53 Inhibitor), poly ADP-ribose polymerase inhibitor, deferoxamine, ethyl pyruvate, activated protein C, insulin, recombinant erythropoietin, hepatocyte growth factor, carbon monoxide release compound, bilirubin, endothelin antagonist, sphingosine 1 phosphate analog, adenosine analog, inducible nitric oxide synthase inhibitor, fibrate, neutrophil gelatinase-associated lipocalin, IL-6 antagonist, C5a antagonist, IL-10, dexmedetomidine, chloroquine (CQ), hydroxychloroquine (HCQ), and α-melanocyte-stimulating hormone.

16. A composition comprising an intracellular Calcium signaling inhibitor and at least a compound for treating acute kidney injury (AKI).

17. The composition of claim 16, wherein said compound is selected from the list consisting of a recombinant human IGF-I (rhIGF-I), atrial natriuretic peptide (ANP), dopamine, caspase inhibitor, minocycline, guanosine and Pifithrin-α (p53 Inhibitor), poly ADP-ribose polymerase inhibitor, deferoxamine, ethyl pyruvate, activated protein C, insulin, recombinant erythropoietin, hepatocyte growth factor, carbon monoxide release compound, bilirubin, endothelin antagonist, sphingosine 1 phosphate analog, adenosine analog, inducible nitric oxide synthase inhibitor, fibrate, neutrophil gelatinase-associated lipocalin, IL-6 antagonist, C5a antagonist, IL-10, dexmedetomidine, chloroquine (CQ), hydroxychloroquine (HCQ), and α-melanocyte-stimulating hormone.

18. A dosing regimen comprising administration to a subject of a compound for treating acute kidney injury (AKI), and administration of an intracellular Calcium signaling inhibitor.

19. (canceled)

20. A pharmaceutical composition comprising a therapeutically effective amount of a compound of claim 9, and a pharmaceutically acceptable excipient.

21. (canceled)

22. (canceled)

23. The method of claim 9, wherein said intracellular Calcium signaling inhibitor is a compound of chemical name N-(5-(7-chloro-2,3-dihydro-[1,4]dioxino[2,3-b]pyridin-6-yl)pyridin-2-yl)-2,6-difluorobenzamide or a pharmaceutically acceptable salt, pharmaceutically acceptable solvate, or pharmaceutically acceptable prodrug thereof.


SEQUENCE LISTING (3 sequences)

SEQ ID NO: 1 - DNA, 1496 bp, Homo sapiens
  source 1..1496: mol_type = unassigned DNA
agcggcgccgcgggcctgcgtgctggggcagcgggcacttcttcgacctcgtcctcctcgtcctgtgcggccggccgggtgaggccgggcccgcgtagggggcagtcggcggctgcctccggcggaggtgcctcgcggcgcccgggccggcccgcgcctcggcggcgtgctccatgcatccggagcccgccccgcccccgagccgcagcagtcccgagcttcccccaagcggcggcagcaccaccagcggcagccgccggagccgccgccgcagcggggacggggagcccccgggggccccgccaccgccgccgtccgccgtcacctacccggactggatcggccagagttactccgaggtgatgagcctcaacgagcactccatgcaggcgctgtcctggcgcaagctctacttgagccgcgccaagcttaaagcctccagccggacctcggctctgctctccggcttcgccatggtggcaatggtggaggtgcagctggacgctgaccacgactacccaccggggctgctcatcgccttcagtgcctgcaccacagtgctggtggctgtgcacctgtttgcgctcatgatcagcacctgcatcctgcccaacatcgaggcggtgagcaacgtgcacaatctcaactcggtcaaggagtccccccatagcgcatgcaccgccacatcgagctggcctgggccttctccaccgtcatcggcacgctgctcttcctagctgaggtggtgctgctctgctgggtcaagttcttgcccctcaagaagcagccaggccagccaaggcccaccagcaagccccccgccagtggcgcagcagccaacgtcagcaccagcggcatcaccccgggccaggcagctgccatcgcctcgaccaccatcatggtgcccttcggcctgatctttatcgtcttcgccgtccacttctaccgctcactggttagccataagactgaccgacagttccaggagctcaacgagctggcggagtttgcccgcttacaggaccagctggaccacagaggggaccaccccctgacgcccggcagccactatgcctaggcccatgtggtctgggcccttccagtgctttggccttacgcccttccccttgaccttgtcctgccccagcctcacggacagcctgcgcagggggctgggcttcagcaaggggcagagcatggagggaagaggatttttataagagaaatttctgcactttgaaactgtcctctaagagaataagcatttcctgttcttccagctccaggtccacctcctgttgggaggcggtggggggccaaagtggggccacacactcgctgtgtcccctctcctcccctgtgccagtgccacctgggtgcctcctcctgtcctgtccgtctcaacctccctcccgtccagcattgagtgtgtacatgtgtgtgtgacacataaatatactcataaggaaaaaaaaaaaaaaaaaaaaaaaaaaaaa

SEQ ID NO: 2 - DNA, 2495 bp, Homo sapiens
  source 1..2495: mol_type = unassigned DNA
ggagagcctgagttggcattcgtataaatgacctgcctggctcccaccatgagtgctgagcttaacgtgcctatcgacccctctgctcctgcctgccctgagccaggccataagggcatggattaccgggactgggtccgccgcagctacctggaactggtcacctctaaccaccactcggtacaggccctgtcgtggcggaagctctacctgagcagggccaagctgaaggcctccagcaggacctccgccctcctctccggctttgccatggtggccatggtggaggtgcagctggagacgcagtaccagtacccgcggccgctgctgattgccttcagcgcctgcaccacggtgctggtggccgtgcacctgttcgccctcctcatcagcacctgcatcctgcccaatgtggaggccgtgagcaacatccacaacctgaactccatcagcgagtccccgcatgagcgcatgcacccctacatcgagctggcctggggcttctccaccgtgcttggcatcctactcttcctggccgaggtggtgctgctctgctggatcaagttcctccccgtggatgcccggcgccagcctggccccccacctggccctgggagtcacacgggctggcaggccgccctggtgtccaccatcatcatggtgcccgtgggcctcatcttcgtggtcttcaccatccacttctaccgctccctggtgcgccacaaaacggagcgccacaaccgcgagatcgaggagctccacaagctcaaggtccagctggacgggcatgagcgcagcctgcaggtcttgtgaggggccgagggccggggctgggagcggccctgtgcccgggagtccgcagaggcggggatttgtcagatgcagacattttgcaaggctgccgggtagttcaagaccaaagttttcctcttgtcttaataccataaggactggatgacttctcctgagatagaaccgtttggttcaatgagggactgtgttgctaagagcgttgggggcaaagccaggctggttccttggcctcggggtttcctgggtcggggacacggtgaagaggctccagcgggacctgcccatcagtcctgggccaggaggggctccaagcagcacccagcggtccgggggagtctcagacccggcatgcgtggctggcagacctgggagagccagggcagggttttgcgttcagagaaggattgccccagagacccgtggtggacttcatgggtgctgagtggcccgtgtgacagtgatgacacgaaggcttcggcgtttgagtgggtgcaggtgcacgccagggcttggtgcttccctgcctggccctggagggagctgggtggcctggcttcaggggaagacaggagccaggacacacgtcagcccagcaggtgtggggggtgctgcagccctcggcagtggggtcaggccctgggggatgtttccaatggtgggcagcctggccaggccggagaagacatgttcacgggcatctatcagatgcccccttgaggaggctgagttatttgagggctgctgcaaagtacgctaggctcaaattctcttttcccagccagagccctggccacacggactcagaggggccaccggggtggggaaaggacccgtccccgaccccccgcagccactggcctccagctctcggccacagaatggcctctaaggctgactcagccgctcccttgggctgtggcagcaggaggcgggggctctggctcaggccccggagcctgtgcagcttgcccatggccctaggcagcgaggggacagcctgggggacttcctgcctaggcaaggtcattggccgggcctggcctgtggatagtggggccaggggccggcccaggccaaatgagtgccctccttgttatgacaccaagtgactacaagggaggcaagacccctccaggcctctcagccgacactgggtcccaccacacacagtgactgtgccgtgcagtgcaggttctggccttttccttgaaggcatctggtagacccgaagccacgctctcgggccgcacatgcacgccgcagcaccagctgccctgagctgcttgtacaaccaaacacctttcccctcttctccagctgtaacctggagagtcagccatgccttgtcttttgttctcataaatagtcactggggccgggcgcagtgactcacgcctgtaatcccagcactttgggaggcctaggtgggcggatcacttgaggtcaggagttcgagaccagcctggccaacatggtgaaaccctgtctctactaaaaaaatacagaaaattagctgggcgtggtggcgggcgcctgtagccccagctacttgggaggctgaggtgggagaatggcaatggcgtgaacccgggaggcagagcttgcagtgagctgagatggcgccactgcactccagcctgggcgacagagccagactcaatctcaaaaaaaaaaaaaaaaaa

SEQ ID NO: 3 - DNA, 2239 bp, Homo sapiens
  source 1..2239: mol_type = unassigned DNA
cgctccggctcctggggctccccgcagacgctgcttttcttgctccactgggggtgcctcttcctgggcgcccgccgcctgcatcctgctcgccctgtctgggaatggggccgcccccgggcttgggccggcccggctggggcccccgaggcgcttccgccccgtagtgaccgcctggtgccgcccccccccaggatgaagggcggcgagggggacgcgggcgagcaggccccgctgaaccctgagggcgagagccctgcaggctcggccacgtaccgggagttcgtgcaccgcggctacctggacctcatgggggccagtcagcactcgctgcgggcgctcagctggcgccgcctctacctcagccgggccaagctcaaagcttccagccgcacgtctgccttgctctcgggcttcgccatggtggccatggtggaggtgcagctggagagtgaccacgagtacccaccaggcctgctggtggccttcagtgcctgcaccaccgtgctggtggctgtgcacctctttgcactcatggtctccacgtgtctgctgccccacattgaagctgtgagcaacatccacaacctcaactctgtccaccagtcgccacaccagagactgcaccgctacgtggagctggcctggggcttctccactgccctgggcacctttctcttccttgctgaagttgtcctggttggttgggtcaagtttgtgcccattggggctcccttggacacaccgacccccatggtgcccacatcccgggtgcccgggactctggcaccagtggctacctcccttagtccagcttccaatctcccacggtcctctgcgtctgcagcaccgtcccaggctgagccagcctgcccaccccggcaagcctgtggtggtggtggggcccatgggccaggctggcaagcagccatggcctccacagccatcatggtacccgtggggctcgtgtttgtggcctttgccctgcatttctaccgctccttggtggcacacaagacagaccgctacaagcaggaactagaggaactgaatcgcctgcagggggagctgcaggctgtgtgagactggtgttagccaccgctcactgcaagcactgcctccctccggggtctgtaagaggccgcaggggcctacagacctcatccccccatcccctggctggagccacttccagtggccactctcaggcagagttcagattcctgcccgcagggtcctctgggctgggccttggggcagctcccacattcccagggattttccccatcagtctgtcccttgggttttgcaagctactctgcacctgggctggcctcagttgaaggatcatgcagtagatagaggggaggcagggagagcttgtgggaccttcagtgctgactttagccaccatttccattcctatacaggatgtgaaggtcagaaggcagccaattgttggtttaattttttttttttttgagacagtctgtttcccaggctggagtgtagtgatacagtcacagctcactgtagcctcgaccttccaggctcaaaagatgctcccaccacagcctcccaggtagtgagtagctggtactacaggtgtgtgctgccacacccgactaatttttttgtagagacggggtttcgctgttcccaggctggtctcaaactcctgggctcaagtgaacctcccgcctcggcctcccaaagtgctgggattcctttctttatttctgtagaatctattttatggttggcattttgggggaagatttcgatgggttccacattcttgctttagttgttgtagagggatttgggtgtttctacccaaggcattggtctagcttttcctacaatgaacctatctttggaggtttaagctccccaccttcccccactgtggtgacctgtggccacttgcagaagggatggtgcctgacccactgccctagccccacgctatgcaccaaacttgttctccccgtcctggtccagggctggggtctttagagactgacagcctctgccccaggcctgagtccttagcaagggttgggtaaggaggttttaagggagaaggtccagtccttagcccttgaaatacaaagctcttctgacactgaatttggatgcaccttgttttatataataaatcgtgtttcacagaaaaaaaaaaaaaaaaaaaaaaaaaaaaaaaaaaaaaaaaaaaaaaaaaaaaaaaaaaaaaaaa